(19) United States
(12) Patent Application Publication
(10) Pub. No.: US 2022/0249525 A1
(43) Pub. Date: Aug. 11, 2022

(54) Fatty Acid Compounds for Prevention and Treatment of Neurodegenerative Disorders

(71) Applicant: T OMEGA AS, Tananger (NO)

(72) Inventors: Rolf Kristian Berge, Bønes (NO); Bodil Bjørndal, Bergen (NO)

(21) Appl. No.: 17/612,797

(22) PCT Filed: May 20, 2020
(86) PCT No.: PCT/NO2020/050133

§ 371 Date: Nov. 19, 2021

(30) Foreign Application Priority Data
May 20, 2019 (NO) 20190650
May 20, 2019 (NO) 20190651

(51) Int. Cl. A61K 31/685; A61K 31/683; A61K 31/201; A61P 25/28

(52) U.S. Cl. CPC A61K 31/685; A61P 25/28; A61K 31/201; A61K 31/683

(57) ABSTRACT

A fatty acid or fatty acid containing compound for use in the prevention and/or treatment of a neurodegenerative disorder, and related novel compounds.

### BACKGROUND

The disclosed embodiments relate to a fatty acid compound or fatty acid containing compound for use in the prevention and/or treatment of neurodegeneration, and relates to novel compounds.

Neurodegenerative diseases (NDs) is characterized by progressive neuronal degeneration and death. These diseases have an increasing prevalence due to longer life expectancy and a larger share of older people in the total world population. NDs are a heterogeneous group of disorders, and often present with dementia (e.g., Alzheimer's disease, AD) or as a movement disorder (e.g., Parkinson's disease, PD). The diseases are mostly idiopathic and develop progressively and irreversibly. Current treatments focus only on reducing symptoms as there are no disease-modifying therapies.

General features of NDs include a selective loss of nerve cells and deposits of abnormal peptides in neurons or associated glial cells. The disorders are therefore often referred to as proteinopathies and include both the misfolding of proteins and their harmful aggregation intra- or extracellularly.

AD is the most common type of cognitive impairment (dementia) in all age groups. It appears mostly sporadic after the age of 65 (late-onset AD, LOAD), but 5-10% of all cases are inherited in an autosomal dominant manner typically before the age of 55 (early-onset AD, EOAD). The cause of AD is not entirely understood, but a pathological hallmark is an accumulation of amyloid-β (Aβ, plaques) mainly in the extracellular space between neurons and the formation of neurofibrillary tangles (NFT) consisting of hyperphosphorylated tau protein intracellularly in neurons. AD is also associated with the loss of neurons and synaptic function, mitochondrial abnormalities and inflammatory responses. In particular, evidence suggests that an accumulation of Aβ contributes to mitochondrial dysfunction through interaction with mitochondrial membranes and proteins. Reversely, it is also proposed that mitochondrial dysfunction in itself causes Aβ-formation and deposition, synaptic degeneration and NFT-formation. The most important risk factor for AD apart from advancing age is being a carrier of a particular variant of the apolipoprotein E gene (APOE). The gene has three alleles, ε-2, ε-3 and ε-4, where the ε-4 variant (APOE4) is associated with AD. In AD-patients, 65-80% carry at least one APOE4 allele. Carriers of two alleles have a 20-fold risk of developing AD. There is no consensus of the role of APOE in AD, but it has been shown to bind and influence the removal of A13 from the brain.

PD is the second most common type of ND after AD and the most common neurodegenerative movement disorder. The prevalence is 1-2% in people over 65, and 5-10% of the cases are familial. The main pathological features are the loss of dopaminergic neurons in the substantia nigra of the midbrain, and the accumulation of Lewy bodies mainly consisting of α-synuclein in the cytoplasm of neurons. α-synuclein is a protein with unknown functions but is associated with presynaptic terminals and may be involved in neurotransmitter release and synaptic plasticity. As in AD, evidence indicates that mitochondrial dysfunction is a central factor in the development of PD. This may include impairment of mitochondrial biogenesis, increased reactive oxygen species (ROS) production, dysfunction in the electron transport chain (ETC) and defective mitophagy, to mention some.

Mitochondrial dysfunction plays an important role in several neurological disorders. The pathogenesis and clinical manifestations arise from the fundamental role of bioenergetics in cell biology. Eventually, cells will die if depleted of ATP. Mitochondrial injury may lead to the release of pro-apoptotic factors (e.g., cytochrome c). Many of the pathways involving mitochondrial dysfunction in AD are also prevalent in the pathogenesis of PD

Thus, the study aimed to investigate the potential effects fatty acids have on brain cells by using the in vitro model SH-SY5Y and to compare it with the HuH-7 cell line serving as a model for liver where fatty acids such as TTA has known effects. A secondary aim was to test different fatty acid-analogs in cell culture.

### Mitochondrial Dysfunction

Mitochondria power cells by generating ATP. The energy required to produce ATP is created by the highly efficient transfer of electrons down a series of carriers (Complexes I-IV) that comprise the electron transport chain (ETC). This reaction is completed by the transfer of electrons to oxygen. However, if this process does not operate properly electrons leak from members of the ETC (Complexes I and III) to oxygen increasing the formation of injurious reactive oxygen species (ROS). The low anti-oxidant capacity and high metabolic activity of neurons render these cells particularly susceptible to ROS-mediated damage. Oxidative injury resulting from mitochondrial dysfunction is a central pathological feature of neurodegenerative disorders such as Parkinson's disease, stroke, Huntington's disease, amyotrophic lateral sclerosis, Alzheimer's disease and multiple sclerosis. Treatments that reduce ROS production by improving mitochondrial function have therefore attracted considerable interest as therapeutics for these disorders, However, clinical development of neuroprotective drugs is hampered by the tremendous cost, long duration, complexity and high failure rate of human efficacy trials. Identification of an acute condition resulting from pathological processes relevant to more common neurodegenerative disorders would mitigate these problems by permitting rapid proof-of-concept to be clearly established in a small group of patients.

Mitochondrial uncoupling protein 3 is a protein that in humans is encoded by the UCP3 gene. UCP3 is a mitochondrial uncoupling protein 3, which is encoded by UCP3. The gene is located in chromosome (11q13.4) with an exon count of 7 (HGNC et al., 2016). Uncoupling protein being a supreme family of mitochondrial anion carrier. Its functions is to separate the oxidative phosphorylation from synthesis of ATP as energy which is anticipated as heat. The uncoupling proteins involves in the transferring of anions from inner mitochondrial membrane to outer mitochondrial membrane, its protein is programmed in a way to protect mitochondria from induced oxidative stress.

### SUMMARY

Disclosed herein are embodiments related to a fatty acid or fatty acid containing compound for use A fatty acid or fatty acid containing compound for use in the prevention and/or treatment of a neurodegeneration disorder, wherein the fatty acid has the general formula (I):

R¹—[Xᵢ—Xᵢ]n-Y (I)

wherein R¹ is;
a C₆-C₂₄ alkene with one or more double bonds and/or with one or more triple bonds, and/or
a C₆-C₂₄ alkyne, or
a C₆-C₂₄ alkyl substituted in one or several positions with one or more compounds selected from the group comprising fluoride, chloride, hydroxy, C₁-C₄ alkoxy, C₁-C₄ alkylthio, C₂-C₅ acyloxy or C₁-C₄ alkyl, and

wherein n is an integer from 1 to 12, and

wherein i is an odd number and indicates the position relative to the α-carbon in Y, and

wherein at least one Xᵢ independent of each other is N, S, or CH₂, and

wherein at least one Xᵢ is N or S, and

wherein Y is CO—COOR₂, CH₂—COOR₂, or CH₂—R4, and wherein R4 is carboxylic acid or a derivate thereof, wherein the derivate is a carboxylic ester, a glyceride or a phospholipid

wherein R₂, if present, represents hydrogen or C1-C4 alkyl,

with the provision that if Xi is S, then one carbon-carbon triple bond or carbon-carbon double bond is positioned between the (ω-1) carbon and the (ω-2) carbon, or between the (ω-2) carbon and the (ω-3) carbon.

In an embodiment is the neurodegenerative disorder present in an individual with patient dementia.

In an embodiment is the neurodegenerative disorder present in an individual with Alzheimer's disease.

In an embodiment is the neurodegenerative disorder present in an individual with movement disorder.

In a preferred embodiment is said fatty acid or fatty acid containing compound a glyceride derived from monoacylglycerols, diacylglycerols or triacylglycerols.

In a preferred embodiment is said fatty acid containing compound a phospholipid derived from lysophospholipids, phosphatidylserines, phosphatidylcholines, phosphatidylethanolamines, phosphatidylinositols (PI), phosphatidic acids or phosphatidylglycerols.

In a preferred embodiment is Xi defined as N.

In a preferred embodiment is Xi defines as N, and R₁ is an alkyne.

In a preferred embodiment is Xi defined as N and R₁ is an alkyne with one triple bond.

In a preferred embodiment is the fatty acid of fatty acid entity Tetradec-12-yn-1-ylglycine.

In a preferred embodiment is the fatty acid of fatty acid entity N-tetradecylglycine.

In a preferred embodiment is the fatty acid of fatty acid entity Tetradecylthioacetic acid.

In a preferred embodiment is the fatty acid of fatty acid entity 2-(tridec-12-yn-ylthio) acetic acid.

In a preferred embodiment comprises R¹ one carbon-carbon triple bond.

In a preferred embodiment comprises R¹ one carbon-carbon double bound.

In a preferred embodiment is the carbon-carbon double bond in a cis configuration.

A second aspect of the disclosure relates to a novel fatty acid or fatty acid containing compound of the general formula (I):

R¹—[Xᵢ—Xᵢ]n-Y (I)
wherein R¹ is;
a C₆-C₂₄ alkene with one or more double bonds and/or with one or more triple bonds, and/or
a C₆-C₂₄ alkyne, or
a C₆-C₂₄ alkyl substituted in one or several positions with one or more compounds selected from the group comprising fluoride, chloride, hydroxy, C₁-C₄ alkoxy, C₁-C₄ alkylthio, C₂-C₅ acyloxy or C₁-C₄ alkyl, and
wherein n is an integer from 1 to 12, and
wherein i is an odd number and indicates the position relative to α-carbon in Y, and
wherein at least one Xᵢ independent of each other is N or CH₂, and
wherein at least one Xᵢ is N, and
wherein Y is CO—COOR₂, CH₂—COOR₂, or CH₂—R4, and wherein R4 is carboxylic acid or a derivate thereof, wherein the derivate is a carboxylic ester, a glyceride or a phospholipid
wherein R₂, if present, represents hydrogen or C1-C4 alkyl,
with the provision that one carbon-carbon triple bond or carbon-carbon double bond is positioned between the (ω-1) carbon and the (ω-2) carbon, or between the (ω-2) carbon and the (ω-3) carbon.

In an embodiment is the compound a glyceride derived from monoacylglycerols, diacylglycerols or triacylglycerols.

In an embodiment is the compound a phospholipid derived from lysophospholipids, phosphatidylserines, phosphatidylcholines, phosphatidylethanolamines, phosphatidylinositols (PI), phosphatidic acids or phosphatidylglycerols.

In an embodiment is R1 an alkyne with one triple bond.

In an embodiment is said compound Tetradec-12-yn-1-ylglycine.

In an embodiment is said compound 1-N-(tridec-12-yn-yl) glycine.

In an embodiment is said compound 2-N-(tridec-12-yn-yl) glycine.

### BRIEF DESCRIPTION OF THE DRAWINGS

Embodiments of the present invention and experimental results will now be described, by way of example only, with reference to the following diagrams wherein:

FIG. 1A shows the effect of the test compounds on viability in cultures with HCC827 cells.

FIG. 1B shows the effects on mitochondrial respiration, by measuring oxygen consumption rates (OCR) by extracellular flux analysis by real-time measurements in HCC827 cell cultures.

FIG. 1C shows that the basal respiratory rate was decreased in a dose-dependent manner in cultures treated with TTA and TDG.

FIG. 1D shows that leak respiration, measured after addition of the ATP synthase inhibitor oligomycin, was significantly increased by 100 μM TTA, 200 μM 2-triple TTA and 30 μM TDG.

FIG. 1E shows that the effects of the compounds on uncoupled respiratory capacity, assessed after addition of CCCP, were similar to the effects on basal respiration.

FIG. 2A shows that both resveratrol and AICAR caused a moderate reduction in resazurin conversion.

FIGS. 2B and 2C show that treatment with AICAR (250 μM or 500 μM) for 5 days caused significant increase in GFP intensity in the HeLaNRF1/c4 reporter cells.

FIGS. 3A-3D show that none of the test compounds was found to alter the GFP expression in the HeLaNRF1/c4 reporter cells.

FIGS. 4A-4F show the gene expression of a panel of factors central for mitochondrial energy metabolism. After this long-term treatment, AICAR caused reduced PGC1α expression (FIG. 4A). The expression of ACOX, which is constitutes a target gene for PPARs, was largely unaffected by the compound treatments (FIG. 4B). No clear effects on HIF1α expression (FIG. 4C). The expression of PDH kinase 1 and 4 (PDK1 and PDK4) and SIRT4 (FIGS. 4D-4F).

FIG. 5 shows the viability of MOLM-13 human acute leukemia cells after 48 hours.

FIG. 6 shows the viability after 72 hours, given modified fatty acids TTA, tr-TTA, TDG (TGH) and tr-TDG (tr-TGH).

FIG. 7 shows the gene expression of carnitine palmitoyl transferase 2 (Cpt2), Cpt1a, and uncoupling protein 3 (Ucp3) in brains of rats fed control diets or diets with 0.4% TTA. Means values are indicated. Statistical different values from control was analysed by one-way ANOVA with Dunnett's multiple comparisons test, *p<0.05, **p<0.01, ***p<0.001.

FIG. 8 shows triacylglycerol (TAG) in livers of C57BL/6 mice given Mildronate (Meldonium), TTA, Mildronate+TTA compared to Control for three weeks. Values are shown as means with standard deviation (n=8-10). One-way ANOVA with Tukey's multiple comparisons test was used to determine significant differences between the intervention groups (***P<0.001).

FIG. 9 shows the results of WST-1 assay after 2 days of treatment with different FAs and controls in a 2-fold dilution series. % absorbance is compared to the mean value of untreated cells in medium only. The x-axis is in a log 2 scale. The data is from one treatment with 2 parallels per FA and control.

FIG. 10 shows the results of WST-1 assay after 4 days of treatment with FAs and controls in a 2-fold dilution series. The % absorbance is based on the absorbance of the mean value of untreated cells in medium only. The x-axis is in a log 2 scale. The data are from two separate experiments with two parallels each and presented as the mean % absorbance of the treatments.

FIG. 11 shows the results of WST-1 assay after 4 days of treatment with FAs and controls in a 2-fold dilution series. The % absorbance is based on the absorbance of the mean value of untreated cells in medium only. The x-axis is in a log 2 scale. The data are from four separate experiments with two parallels each and presented as the mean % absorbance of the treatments.

FIG. 12 shows the effect of TTA on selected PPAR target genes versus control in SH-SY5Y cells after 4 days of treatment. Gene expression is shown as relative values using the 2^{-ΔΔCt} method with HPRT1 as the reference gene.

FIG. 13 shows effects of different FAs versus control in SH-SY5Y cells after 4 days of treatment. The data were analyzed using the 2^{-ΔΔCt} method with 18 S as a reference gene.

FIG. 14 shows the effect on selected genes after FA treatment over 4 days in HuH-7.

FIG. 15 shows the effects of treatment with FAs dissolved in DMSO in HuH-7 cells after 4 days of treatment. The analysis was performed using the 2^{-ΔΔCt} method relative to HPRT1 and all samples were calibrated against CTR (cells only in growth medium).

FIG. 16 shows the effects of treatment with FAs dissolved in DMOS in SH-SY5Y cells after 4 days of treatment. The analysis was performed using the 2^{-ΔΔCt} method relative to HPRT1 and all samples were calibrated against CTR (only growth medium).

FIGS. 17A-17I show the effect of treatment with TTA complexed with FBS at 125 μM in HuH-7 cells. Values shown are mean values of two parallels with SDs for TTA complexed with FBS. PA and CTR were only performed as one parallel. The analysis was performed using the 2^{-ΔΔCt} method relative to HPRT1.

FIG. 18 shows the effects of TTA and PA on SH-SY5Y-cells after 24 hours of treatment. The analysis was performed using the 2^{-ΔΔCt} method relative to HPRT1. Mean values of two parallels with SDs are shown.

FIG. 19 shows gene expression of Angptl4, Cpt-2, Pdk4 and Ucp3 in brain after the supplementation of TTA in low (0.4%) and high (0.75%) dose in diet compared to control using the 2^{-ΔΔCt} method relative to HPRT1. Mean values are indicated (n=5). Statistical difference in values was calculated using the one-way ANOVA with Dunnett's multiple comparisons test, * p<0.05, ** p<0.01, *** p<0.001, **** p<0.0001.

FIG. 20 shows gene expression of selected PPAR target genes, genes for PPARα and PPARβ/♦ and PGC-1α. Mean values are indicated for the treatments (n=8). Statistical differences were calculated using the Student's t-test, * p<0.05, ** p<0.01, *** p<0.001, **** p<0.0001.

FIG. 21 shows gene expression of PPARα and PPARβ/δ and PGC-1α. Mean values are indicated for the treatments (n=8). Statistical differences were calculated using the Student's t-test, * p<0.05, ** p<0.01, *** p<0.001, **** p<0.0001.

FIGS. 22A-22D show body weight, feed intake and weight of white adipose tissue (WAT) depots in mice fed a high-fat diet (Control) or high-fat diets supplemented with N-TTA (750 mg/kg body weight or triple-N-TTA (350 mg/kg body weight) for 4 weeks. Means values with standard deviations are indicated (n=5-7, feed intake n=2). Statistical difference from Control was analyzed where relevant by one-way ANOVA with Dunnett's multiple comparisons test (*p<0.05, **p<0.01, ***p<0.001).

FIG. 23 shows the correlation between weight gain and in vitro hepatic beta-oxidation in mice fed a high-fat diet (Control-blue dots) or high-fat diets supplemented with N-TTA (750 mg/kg body weight, purple dots) or triple-N-TTA (350 mg/kg body weight, red dots) for 4 weeks. Person correlation coefficient (r) was calculated using two-tailed p-value and a 95% confidence interval.

FIGS. 24A-24E show the plasma glucose and cholesterol levels in mice fed a high-fat diet (Control) or high-fat diets supplemented with N-TTA (750 mg/kg body weight or triple-N-TTA (350 mg/kg body weight) for 4 weeks. Means values with standard deviations are indicated (n=5-7). Statistical difference from Control was analyzed by one-way ANOVA with Dunnett's multiple comparisons test (*p<0.05, **p<0.01, ***p<0.001).

### DETAILED DESCRIPTION

Abbreviations used in the disclosure and drawings are as follows:

| | |
|---|---|
| TTA: | Tetradecylthioacetic acid |
| Tr-n-TTA or tr-TDG | Tetradec-12-yn-1-ylglycine hydrochloride |
| 2-tr-TTA | 2-(Tetradec-12-yn-1-ylthio)acetic acid |
| 1-tr-TTA | 1-(Tetradec-12-yn-1-ylthio)acetic acid |
| TDG or N-TTA | N-tetradecylglycine |
| 2-triple TDG | N-(tridec-12-yn-yl) glycine |

### EXPERIMENTAL SECTION

### Example 1

### Preparation of N-Tetradecylglycine (N-TTA)

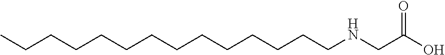 [SMILES: CCCCCCCCCCCCCCNCC(=O)O]

Structure of N-tetradecylglycine (termed N-TTA or TDG in the present application).

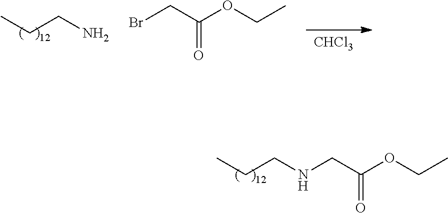

Ethyl bromoacetate (7.2 mL, 65 mmol) dissolved in chloroform (50 mL) was added dropwise to a solution of tetradecylamine (26.32 g, 123 mmol) in chloroform (250 mL) over approximately 30 minutes. After the addition was completed the reaction was stirred for an additional hour at ambient temperature.

The crude reaction mixture was reduced under reduced pressure and the product was purified by column chromatography on silica using a gradient of methanol in dichloromethane.

Yield: 14.96 g, 49.9 mmol.

¹H NMR (CDCl₃, 400 MHz): 4.17 (q, 7.1 Hz, 2H), 3.38 (s, 2H), 2.62-2.53 (m, 2H), 1.45 (m, 2H), 1.34-1.18 (m, 25H), 0.85 (t, 6.8 Hz, 3H)

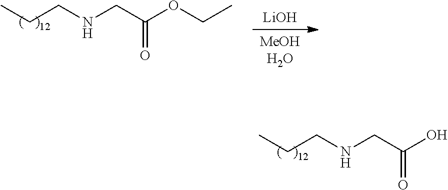

Ethyl tetradecylglycinate (19.83 g, 66.2 mmol) was dissolved in methanol (400 mL) and water (80 mL). Lithium hydroxide monohydrate (11.07 g, 264 mmol) was added and the reaction mixture was stirred over night at ambient temperature.

Formic acid (15 mL) was added dropwise to the reaction mixture and the reaction mixture was reduced under reduced pressure and the product was purified by column chromatography on reversed phase silica using a gradient of acetonitrile in water. Yield: 10.20 g (37.6 mmol).

¹H NMR (MeOH-d₄, 400 MHz): 3.49 (s, 2H), 3.03-2.90 (m, 2H), 1.73-1.63 (m, 2H), 1.43-1.23 (m, 22H), 0.90 (t, 6.8 Hz, 3H)

### Example 2—Preparation of Tetradec-12-yn-1-ylglycine Hydrochloride (tr-N-TTA)

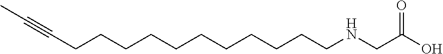 [SMILES: CC#CCCCCCCCCCCCNCC(=O)O]

Structure of tetradec-12-yn-1-ylglycine (termed tr-N-TTA or tr-TDG in the present application)

### Tert-Butyl tetradec-12-yn-1-ylglycinate (AKB:DP-5:61-EH-1)

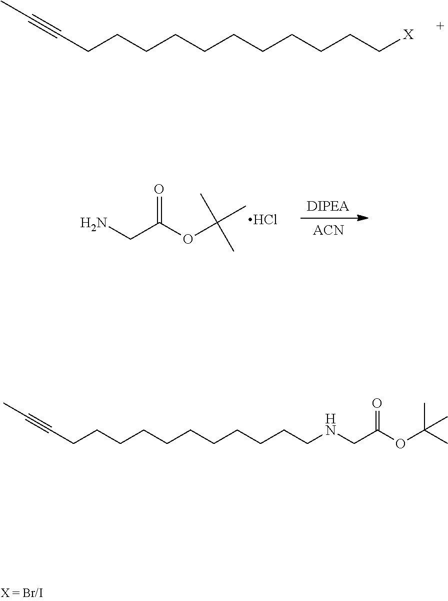

A mixture of bromo/iodotetradec-2-yne (45 g, 146 mmol) and glycine t-butyl ester hydrochloride (26.9 g, 161 mmol) in ACN, 600 ml, was added DIPEA (63.6 ml, 365 mmol) and the reaction mixture was refluxed for 4 hours. After cooling to room temperature, the mixture was concentrated under reduced pressure. Flash chromatography on silica gel eluting with heptane/EtOAc (95:5)-(70:30)-(65:35) afforded 13 g (28%) of the title compound as a yellow oil and 19 g of the starting material as bromotetradec-2-yne. ¹H NMR (400 MHz, CDCl₃) δ 3.26 (s, 2H), 2.55 (t, J=7.2, 2H), 2.16-1.92 (m, 2H), 1.75 (t, J=2.5, 3H), 1.54-1.38 (m, 14H), 1.24 (s, 13H).

### Tert-Butyl tetradec-12-yn-1-ylglycinate (AKB:DP-5:61-EH-2)

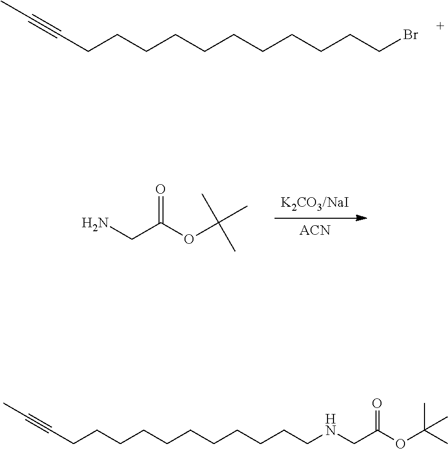

A mixture of bromotetradec-2-yne (13.6 g, 49.9 mmol) and glycine t-butyl ester hydrochloride (9.2 g, 54.9 mmol) in CAN, 200 ml, was added K₂CO₃ (17.3 g, 125 mmol) and NaI (7.5 g, 50 mmol) and refluxed overnight. The reaction mixture was cooled to room temperature, filtered and concentrated under reduced pressure. Flash chromatography on silica gel eluting with heptane/EtOAc (95:5)-(70:30)-(65:35) afforded 5.2 g (32%) of the title compound as a yellow oil and 13.8 g of the starting material.

### Tetradec-12-yn-1-ylglycine Hydrochloride (EH:DP-4:82)

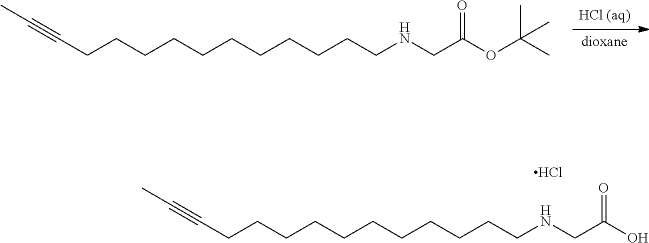

A mixture of tert-butyl tetradec-12-yn-1-ylglycinate (25.8 g, 79.7 mmol) in dioxane, 300 ml, was added 6 M HCl (80 ml) and stirred at room temperature overnight before it was stirred at 55° C. for 6 hours. The reaction mixture was cooled to room temperature and stirred overnight. Precipitated product was filtered off and washed with EtOAc, 200 ml, and dried under reduced pressure to afford 22 g (91%) as a colorless powder.

¹H NMR (400 MHz, DMSO-d6) δ 9.27 (bs, 1H), 3.80 (s, 2H), 2.96-2.78 (m, 2H), 2.57-2.43 (m, 2H), 2.19-1.99 (m, 2H), 1.71 (t, J=2.5, 3H), 1.63 (s, 2H), 1.49-1.14 (m, 14H).

¹³C NMR (101 MHz, DMSO-d6) δ 167.92, 79.28, 75.58, 46.70, 46.69, 28.89, 28.85, 28.72, 28.49 (2C), 28.44, 28.23, 25.89, 25.10, 18.01, 3.07.

MS (pos) 290[M-HCl+Na]⁺

### Example 3—Preparation of 2-tr-TTA

### 2-(Tridec-2-yn-1-yloxy)tetrahydro-2H-pyran (AKB:TM-1:57)

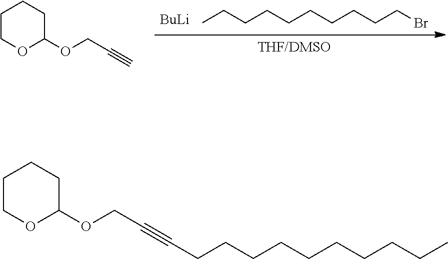

A mixture of 2-(prop-2-yn-1-yloxy)tetrahydro-2H-pyran (67.5 ml, 480 mmol) in dry THF (200 ml) was cooled to 0° C. under N₂-atmosphere before BuLi 1.6 M in hexanes (300 ml, 480 mmol) was added drop wise. 1-Bromodecane (100 ml, 483 mmol) was added followed by DMSO (1000 ml). The cooling bath was removed and the slurry was stirred for 220 minutes. The reaction mixture was cooled to 0° C. before water (250 ml) was added drop wise. Diethyl ether (600 ml) was added and the phases was separated. The organic phase was washed with a (1:1) mixture of water/brine (400 ml×4), dried (Na₂SO₄), filtered and concentrated under reduced pressure. Dry-flash chromatography on silica gel eluting with heptane-heptane:EtOAc (100:1) afforded 88.18 g (65%) of the title compound. ¹H NMR (200 MHz, CDCl₃) δ 4.80-4.77 (m, 1H), 4.40-4.02 (m, 2H), 3.95-3.70 (m, 1H), 3.55-3.44 (m, 1H), 2.31-2.06 (m, 2H), 1.99-1.05 (m, 22H), 0.85 (t, J=6.2, 3H).

### Tridec-2-yn-1-ol (AKB:TM-1:59)

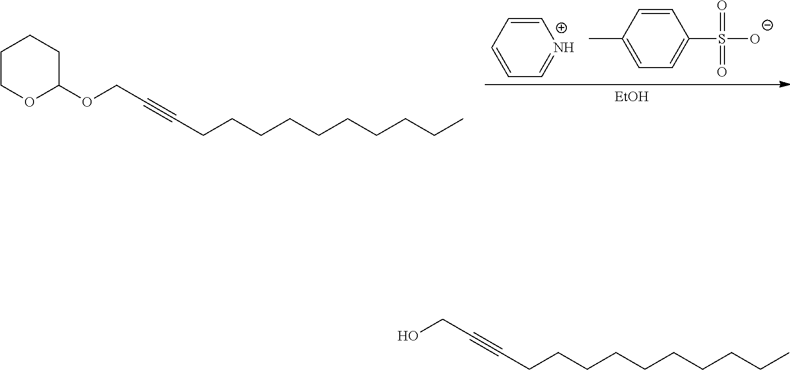 [SMILES: "EtOH" ; CCCCCCCCCCC#CCOC1CCCCO1 ; c1cc[nH+]cc1 ; Cc1ccc(S(=O)(=O)[O-])cc1 ; CCCCCCCCCCC#CCO]

A mixture of 2-(Tridec-2-yn-1-yloxy)tetrahydro-2H-pyran (AKB:TM-1:57) (85.21 g, 303.8 mmol) and PPTS (9.6 g, 38.2 mmol) in EtOH (770 ml) was stirred at 50° C. for 18 hrs and concentrated under reduced pressure. The residue was diluted with CH₂Cl₂ (500 ml) and washed with water (200 ml). The water phase was extracted with CH₂Cl₂ (500 ml). The combined organic phase was dried (Na₂SO₄), filtered and concentrated under reduced pressure. TLC showed remaining starting material. A mixture of the residue and PPTS (7.03 g, 28 mmol) in EtOH (600 ml) was stirred for 17 hrs at 50° C. and concentrated under reduced pressure. The residue was diluted with CH₂Cl₂ (500 ml) and washed with water (200 ml). The water phase was extracted with CH₂Cl₂ (500 ml). The combined organic phase was dried (Na₂SO₄), filtered and concentrated under reduced pressure. Dry-flash chromatography on silica gel eluting with heptane:EtOAc (100:1)-(95:5)-(80:20) afforded 46.06 g (77%) of the title compound as a colorless waxy solid. ¹H NMR (200 MHz, CDCl₃) δ 4.27-4.21 (m, 2H), 2.23-2.15 (m, 2H), 1.65-1.25 (m, 17H), 0.90-0.82 (m, 3H).

### Tridec-12-yn-1-ol (AKB:TM-1:63)

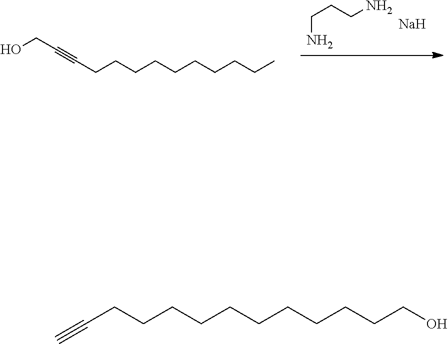

Sodium hydride 60% dispersion in mineral oil (38.82 g, 970.5 mmol) in 1,3-diaminopropane (500 ml) was stirred at 70° C. for 1 hr. The mixture was cooled to room temperature before a solution of tridec-2-yn-1-ol (AKB:TM-1:59) (23.95 g, 122 mmol) in 1,3-diaminopropane (250 ml). The reaction mixture was stirred at 55° C. under N₂-atmosphere for 20 hrs. The mixture was cooled in an ice-bath and water 1000 ml was added. The mixture was extracted with diethyl ether (500 ml×4), washed with 1 M HCl (500 ml), water (500 ml) and brine (300 ml), dried Na₂SO₄, filtered and concentrated under reduced pressure. Dry-flash chromatography on silica gel eluting with heptane-heptane:EtOAc (95:5)-(80:20) afforded 19.76 g (83%) of the title compound. ¹H NMR (200 MHz, CDCl₃) δ 3.62 (dd, J=11.7, 6.4, 2H), 2.16 (td, J=6.9, 2.6, 2H), 1.91 (t, J=2.6, 1H), 1.70-1.05 (m, 18H).

### 13-Bromotridec-1-yne (AKB:TM-1:65)

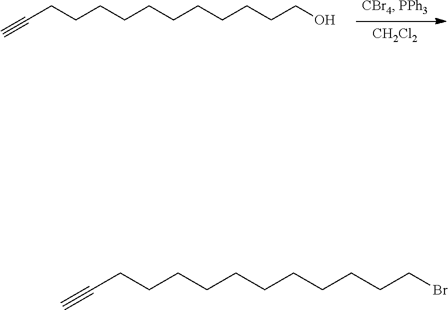

A solution of tridec-12-yn-1-ol (35.27 g, 180 mmol) in dry CH₂Cl₂ (700 ml) was cooled to 0° C. before addition of triphenylphosphine (51.86 g, 197.7 mmol) followed by tetrabromomethane (65.62 g, 197.9 mmol). The reaction mixture was stirred at 0° C. under N₂-atmosphere for 2 hrs. Silica gel was added and the mixture was concentrated under reduced pressure. Dry-flash chromatography on silica gel eluting with heptane afforded 45.55 g (98%) of the title compound as a colorless liquid which solidified upon storage in the freezer. ¹H NMR (200 MHz, CDCl₃) δ 3.38 (t, J=6.8, 2H), 2.16 (td, J=6.9, 2.6, 2H), 1.91 (t, J=2.6, 1H), 1.81 (dd, J=14.7, 6.8, 2H), 1.62-1.11 (m, 16H).

### 14-Bromotetradec-2-yne (AKB:TM-1:67)

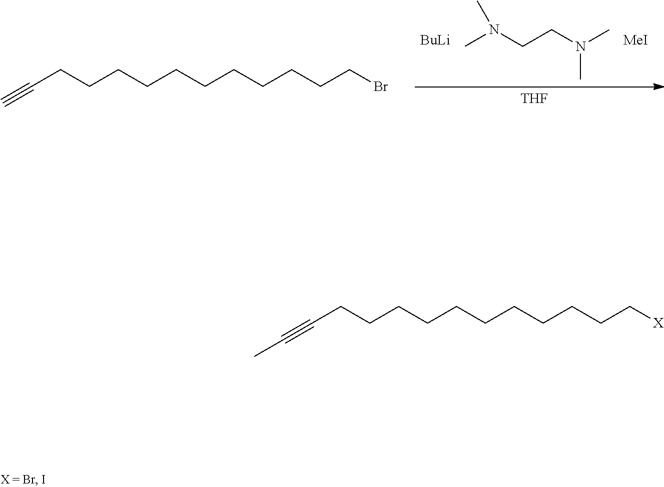

A solution of 13-bromotridec-1-yne (AKB:TM-1:65) (44.68 g, 172.4 mmol) in dry THF (500 ml) was cooled to −10° C. under N₂-atmosphere before BuLi 1.6 M in hexanes (118.5 ml, 189.6 mmol) was added drop wise. The reaction mixture was stirred for 10 minutes before TMEDA (56.5 ml, 376.3 mmol) was added drop wise followed by drop wise addition of methyl iodide (57 ml, 915.6 mmol). A white solid precipitated and extra THF was added in order to stir the reaction mixture. The cooling bath was removed and the reaction mixture was stirred for 18 hrs. Water (500 ml) was added and the phases were separated. The water phase was extracted with diethyl ether (500 ml×2), washed with 1 M HCl (aq) (300 ml), dried (Na₂SO₄), filtered and concentrated under reduced pressure to afford the crude title compound as a mixture of the bromo- and iodo-compound.

### 2-(Tetradec-12-yn-1-ylthio)acetic Acid (AKB:TM-1:69/GH:DP-3:42)

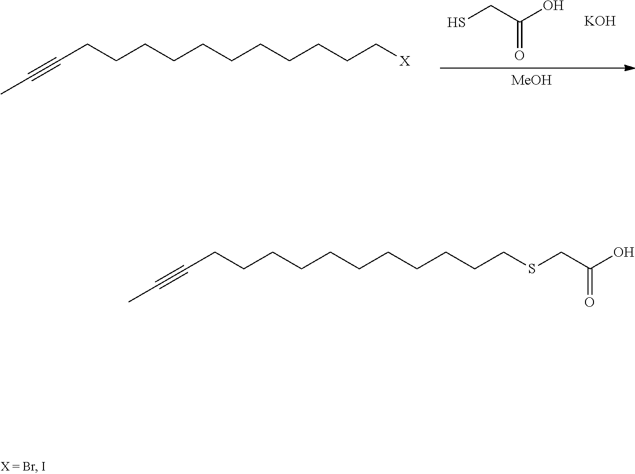

Potassium hydroxide (25.05 g, 446 mmol) was dissolved in MeOH (270 ml) before a solution of 2-mercaptoacetic acid (14 ml, 201.4 mmol) in MeOH (270 ml) was added drop wise. The reaction mixture was stirred for 10 minutes before 14-bromotetradec-2-yne/14-iodotetradec-2-yne (AKB:TM-1:67) (49.74 g) was added drop wise. The 14-bromotetradec-2-yne/14-iodotetradec-2-yne flask was washed out with MeOH (100 ml). The reaction mixture was stirred at 50° C. for 16 hrs, cooled to 0 □C and 1 M and 6 M HCl (aq) was added to pH 1-2 and water 250 ml was added. The mixture was extracted with diethyl ether (1000 ml×2), dried (MgSO₄), filtered and concentrated under reduced pressure. Recrystallization from heptane/EtOAc afforded 22.9 g of the title compound as a light yellow solid. The mother liquor was dissolved in diethyl ether and precipitated with heptane to afford another 10.8 g of the title compound. Total yield 33.7 g (69% from 13-bromotridec-1-yne). ¹H NMR (400 MHz, CDCl₃) δ 11.58 (s, 1H), 3.18 (s, 2H), 2.65-2.51 (m, 2H), 2.06-2.02 (m, 2H), 1.71 (t, J=2.6, 3H), 1.61-1.48 (m, 2H), 1.42-1.36 (m, 2H), 1.25 (d, J=36.2, 14H). MS (neg): 283 [M-H]⁻

### Example 4—Preparation of 1-tr-TTA

1-tr-TTA was obtained in a similar process as described in example 3, but the third last step can be omitted.

### Example 5—Cytoprotective Effects, and Effect on Mitochondrial Function of Modified Fatty Acids

This example shows that a triple bond in omega-2 position of 3-thia and 3-nitro fatty acids have cytoprotective effects in cultured cells

The fatty acid analogues Tetradecythioacetic acid (TTA), 2.2-(tridec-12-yn-yl) thioacetic acid, 2-tr-TTA, N-tetradecylgylcine (TDG), N-(tridec-12-yn-yl) glycine (2-tr-TDG), 2-(ethylthio) pentanoic acid and Ethylthioacetic acid were dissolved in DMSO (Sigma-Aldrich); Other compounds were; Resveratrol (Sigma-Aldrich) dissolved in demethylsulfoxide (DMSO) and 5-aminoimidazole-4-carboxamide-1-β-D-ribofuranoside, AICAR (Toronto Research Chemicals #A611700) dissolved in media.

Non-small cell lung cancer cells, HCC827 (ATCC) were cultured in RPMI-1640 Glutamax medium (Sigma-Aldrich St. Louis, Mo., USA), under conventional culture conditions. Media was supplemented with 10% Fetal Bovine Serum (FBS), 1% glutamine and 1% streptomycin/penicillin (Sigma-Aldrich). HeLaNRF1/c4 reporter cells were cultured in Dulbecco's Modified Eagle's Medium (DMEM) supplemented with 10% FBS, 2 mM L-Glutamine, 100 u/ml penicillin/streptomycin (Sigma-Aldrich,). Incubation was done at 37° C. in 5% CO₂.

Cell viability/proliferation was determined by the resazurin conversion assay. A total of 4000 cells were seeded in each of the 96-well plates in 100 μl of medium. After 3 hours of incubation, 100 μl of fresh medium with respective concentration of the desired compound or controls were added. Resazurin solution (Sigma-Aldrich) was added (10%, v/v in PBS) after 1 or 3 day treatment, followed by 3 hour incubation at 37° C. and 5% CO₂. Resazurin fluorescence (excitation, 540 nm; emission, 590 nm) was measured and measurements were corrected for background signals. Each treatment condition had a minimum of a triplicate wells. Relative viability was calculated from percent fluorescence relative to DMSO-treated control wells.

### Mitochondria Respiration Assays

Mitochondria function was analyzed by measuring oxygen consumption rate (OCR) by extracellular flux analysis in a Seahorse XFe96 instrument (Agilent Seahorse XF Technologies). The measurements were performed in DADA basic media without phenol red, supplemented with L-glutamine (2 mM), pyruvate (2 mM), and sodium chloride, NaCl (32 mM) (Sigma-Aldrich). Respiratory rates were measured following sequential additions of glucose (10 mM), oligomycin, carbonyl cyanide m-chlorophenyl hydrazine (CCCP) and a mixture of rotenone (1 μM) and antimycin A (1 μM). Before carrying out the measurements for each cell model, optimizations were done for; cell number, CCCP, and oligomycin concentrations. For the HCC 827 cell model optimal conditions were; 30,000 cells/well, 0.5 μM CCCP, and 3 μM Oligomycin; and in HeLaNRF1/c4 reporter cell model; 20,000 cells/well, 1 μM CCCP and 3 μM Oligomycin. The OCR measured initially (before any additions) represented the rate ‘basal respiration.’ The addition of ATP synthase inhibitor oligomycin provided oxygen consumption independent of ATP production (‘leak activity’). Maximal respiration (also referred to as ‘respiratory capacity’) was measured upon addition of the uncoupler CCCP. Finally, the addition of respiratory chain complex I and III inhibitors rotenone and antimycin A, respectively, revealed non-mitochondrial respiration, which was subtracted as background. After the extracellular flux analysis the analysis plates were washed with Phosphate Buffered Saline (PBS) and frozen at −80° C. The BCA assay (Pierce BCA protein assay kit, Thermo Fisher Scientific, Waltham, Mass. USA) was employed to measure the protein content in the wells. To evaluate the effects of the different treatments, the respiratory rates from compound-treated wells were normalized to protein content, and calculated as percentage of the average respiratory rate of the DMSO-treated control wells. All treatments were performed with 6-8 replicate wells.

### Mitochondrial Biogenesis

The HeLaNRF1/c4 reporter cells were employed to investigate effects of the modified fatty acids on NRF1-regulated mitochondrial biogenesis. The cells (250,000) were seeded in T75 flasks and incubated for 3 hours to allow attachment. The compounds were dissolved in DMSO and diluted in culture medium to provide the final concentration. DMSO at a concentration equivalent to the highest compound concentration was added to control cultures. Resveratrol and AICAR were used as positive controls. Resveratrol has not previously been tested in this reporter model, but we found it relevant to include it for purposes of experimental and mechanistic comparisons. After 3 days of treatment the media was replaced with fresh media containing the corresponding compound. On day 5, the cells were harvested and the cultures split into three fractions: One fraction was used for detection of mitoGFP expression by flow cytometry, to assess effects on NRF-1 regulated mitochondrial biogenesis. Flow cytometry analysis was performed as previously described [29]. Another fraction was used for extraction of RNA and subsequent gene expression analysis using quantitative PCR (see below). The last fraction was employed to investigate effects on mitochondrial respiratory function by extracellular flux analysis (described above). The experiment was replicated three times.

### Gene Expression Assays

Total RNA was extracted from harvested cells (RNAeasy kit Qiagen, Hilden, Germany). The RNA content was quantified on Nanodrop 1000 Spectrophotometer (Thermo Scientific). From 1 μg total RNA, cDNA was synthesized using High-Capacity Reverse Transcription Kit (Applied Biosystems, Carlsbad, Calif., US). Gene quantification was done using TaqMan probes for gene expression assays (Applied Biosystems). Probes used were: PGC1α (PPARG coactivator 1α), Hs00173304_m1; HIF1α (hypoxia inducible factor 1α), Hs00153153_m1; PDK1 (pyruvate dehydrogenase kinase 1), Hs01561847_m1; PDK4, (pyruvate dehydrogenase kinase 4), Hs01037712_m1; SIRT4 (sirtuin 4) Hs01015516_g1; ACOX1 (acyl-CoA oxidase 1) Hs01074241_m1; as reference genes, we used; Eukaryotic 18S rRNA Hs99999901_s1; actin beta Hs99999903_m1. Fold change was calculated as geometric mean of ΔΔCt of the two reference genes.

### Statistical Analysis

Difference in means among groups were compared using Dunnett's multiple comparison test (a two-sided students T-test) while statistical significance was indicated at p<0.05.

### Short-Term Effects on Cell Viability and Mitochondrial Function

The compounds of interest in this study included TTA and its derivative with triple bond in omega-2 position, 2-(tridec-12-yn-ylthio) acetic acid (2-tripleTTA); the corresponding compounds where the sulphur atom is replaced by a nitrogen atom, N-tetradecylglycine (TDG) and N-(tridec-12-yn-yl) glycine (2-triple TDG); the sulphur-containing valproic acid derivative 2-(ethylthio)pentanoic acid; and the short chain sulphur containing derivative ethylthioacetic acid.

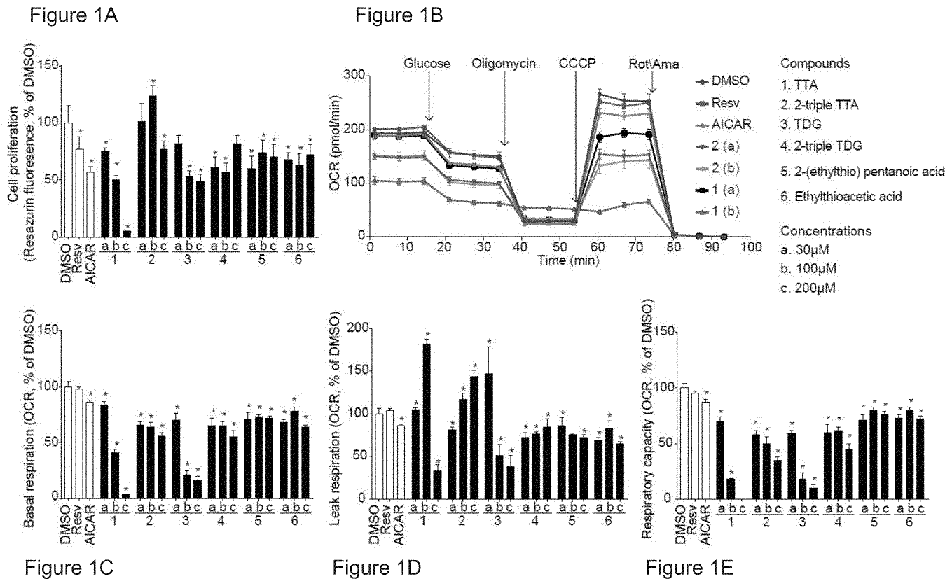

First, we investigated the effect of the test compounds on viability in cultures with HCC827 cells, using the resazurin conversion assay (FIG. 1A). Severe cytotoxicity was seen only with the highest dose (200 μM) TTA, however all compounds, except 2-triple TTA, demonstrated a moderate effect at a concentration of 100 μM which was comparable to treatment with 25 μM resveratrol or 250 μM AICAR. For 2-triple TTA, a higher concentration was required (200 μM) to obtain a moderate antiproliferative effect. Based on this experiment, with three different concentrations of the compounds, a dose-dependent pattern of antiproliferative effects was found for TTA and TDG, but not their respective derivatives with triple bond in omega-2 position (2-triple TTA and 2-triple TDG).

In order to investigate effects on mitochondrial respiration, we measured oxygen consumption rates (OCR) by extracellular flux analysis. Representative examples of real-time measurements in HCC827 cell cultures are shown in FIG. 1B. The basal respiratory rate was decreased in a dose-dependent manner in cultures treated with TTA and TDG (FIG. 1C), consistent with the antiproliferative effects (FIG. 1A). With the other compounds, there was approximately 30-% reduction compared to control, and the effect was not affected by increased concentration.

There was little or no effect of resveratrol or AICAR on basal respiratory rate, compared to control. Leak respiration, measured after addition of the ATP synthase inhibitor oligomycin, was significantly increased by 100 μM TTA, 200 μM 2-triple TTA and 30 μM TDG (FIG. 1D). This indicates that these treatments induce mitochondrial uncoupling. Higher concentrations of TTA and TDG resulted in reduced leak respiration, most likely due to the general collapse of respiratory activity, consistent with cytotoxic effects. Also for the other treatments, the effect on leak respiratory rate agreed with a general reduction in mitochondrial respiration. The effects of the compounds on uncoupled respiratory capacity, assessed after addition of CCCP, were similar to the effects on basal respiration (FIG. 1E).

There was, however, a stronger dose-dependent effect of 2-triple TTA on respiratory capacity compared to basal respiration. In summary, TTA and TDG had similar effects on cell proliferation/viability and mitochondrial function, and at relatively high dosage both had cytotoxic effects in the cultured cells. The harmful effects were significantly reduced in cultures treated with the respective compounds with triple bond in omega-2 position, i.e. 2-triple TTA and 2-triple TDG.

### Mitochondrial and Metabolic Adaptations

In order to investigate if some of the compounds induce metabolic adaptations involving mitochondrial biogenesis, we employed the HeLaNRF1/c4 reporter cells in which activation of the transcription factor NRF1 triggers expression of GFP. Since adaptations of mitochondrial biogenesis are known to require some days to develop, we used a relatively low dosage (30 μM and 60 μM) of the compounds, to allow longer treatment period compared to the previous short term experiments.

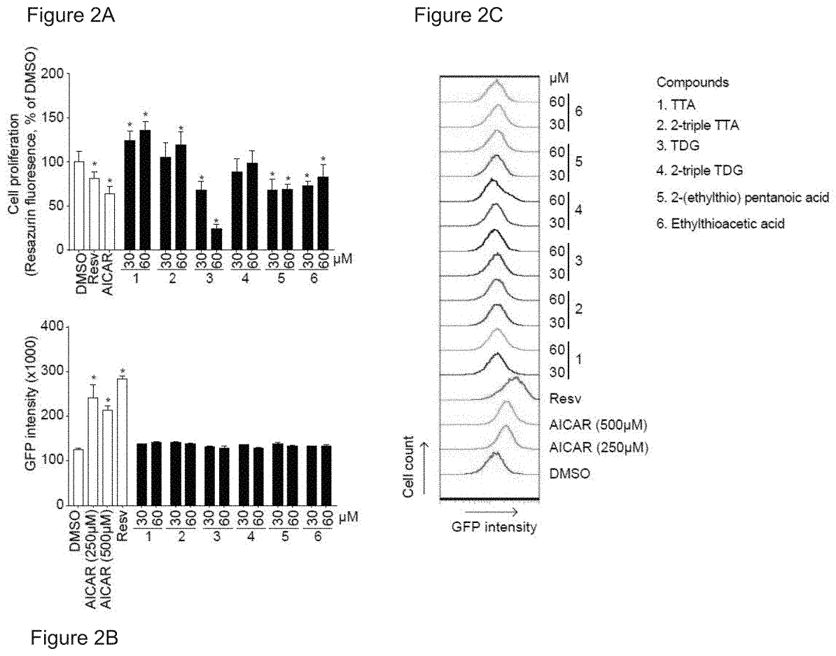

Both resveratrol and AICAR caused a moderate reduction in resazurin conversion under these conditions (FIG. 2A), consistent with AMPK-mediated downregulation proliferation. TTA treatment caused a small increase in resazurin conversion at this dosage, but there were little or no effect of 2-triple TTA. TDG caused a dose-dependent reduction, whereas no significant effect was found for 2-triple TDG. Treatment with 2-(ethylthio) pentanoic acid or ethylthioacetic acid caused 30-40% reduction in resazurin conversion with the tested dosages.

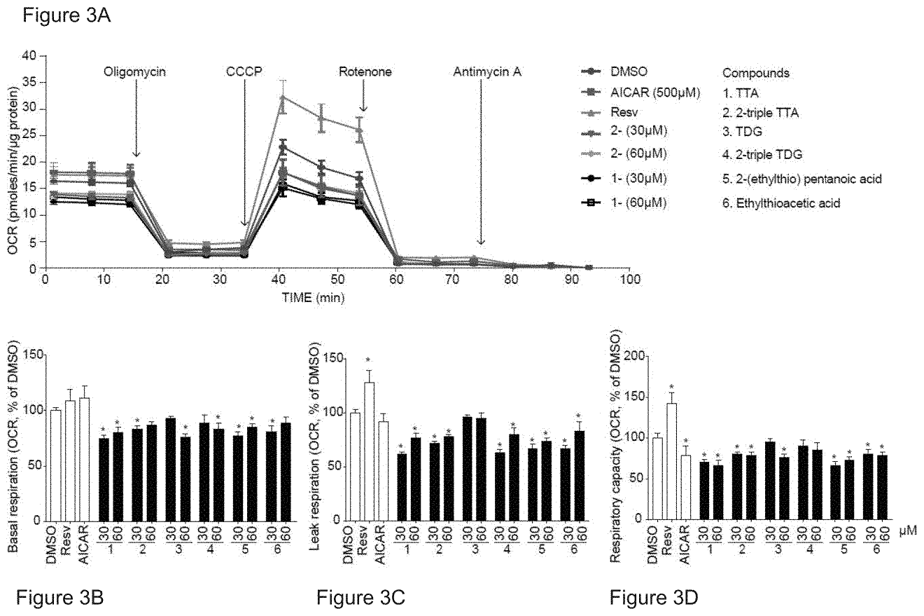

Treatment with AICAR (250 μM or 500 μM) for 5 days caused significant increase in GFP intensity in the HeLaNRF1/c4 reporter cells (FIGS. 2B and 2C), consistent with previous findings. A similar effect was also seen with resveratrol (25 uM), which has previously been reported to activate mitochondrial biogenesis. However, none of the test compounds was found to alter the GFP expression in the HeLaNRF1/c4 reporter cells, suggesting that these treatments do not trigger a typical energy stress response involving NRF1-regulated mitochondrial biogenesis. These data were compatible with the data from subsequent analysis of mitochondrial respiratory rates (FIGS. 3A-3D). All treatments caused a consistent 20-30% reduction in basal respiratory rate and respiratory capacity. With this dosage, the leak respiratory rated did not indicate mitochondrial uncoupling and cytotoxic effects (data not shown). Despite the finding that both AICAR and resveratrol induced mitochondrial biogenesis, the uncoupled respiratory capacity (CCCP) was only increased in the AICAR-treated cells, without affecting the basal respiratory rate (FIG. 3D).

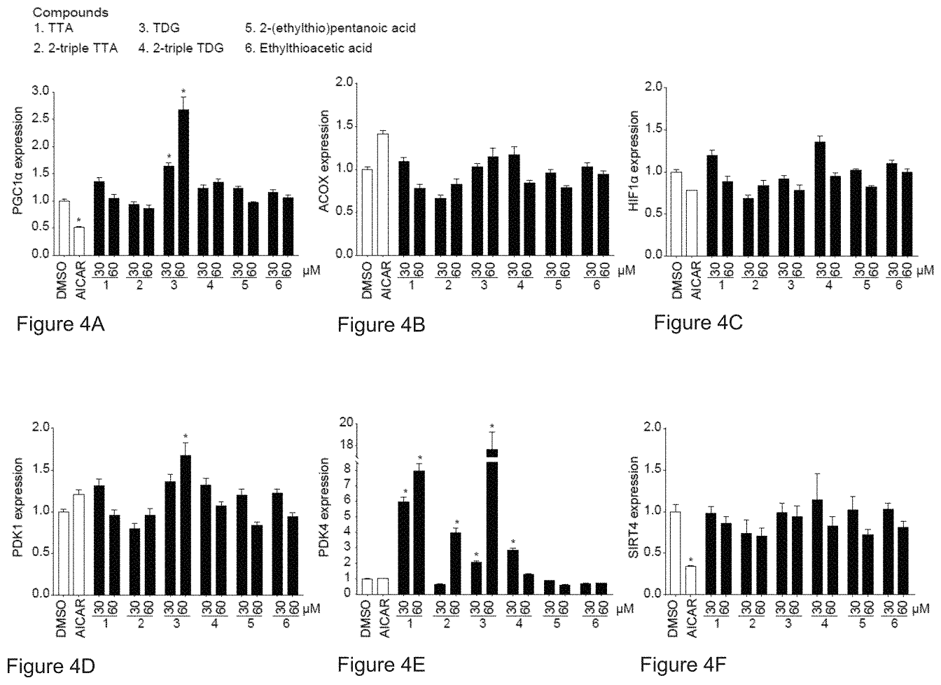

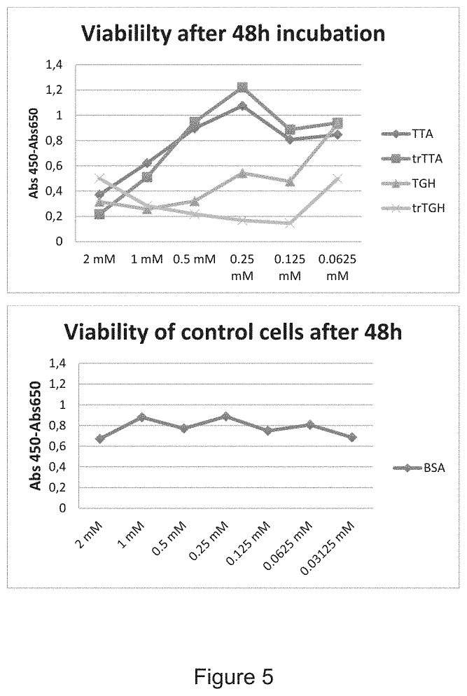

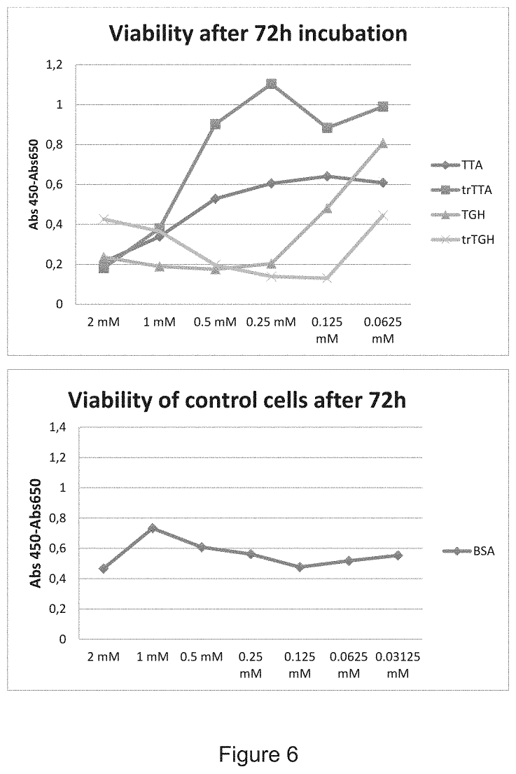

In order to investigate potential effects on metabolic regulation, we measured gene expression of a panel of factors central for mitochondrial energy metabolism (FIGS. 4A-4F). After this long-term treatment, AICAR caused reduced PGC1α expression (FIG. 4A). The test compounds had little or no effect on PGC1α expression compared to the control, apart from cultures treated with TDG, where there was a significant increase. Interestingly, the expression level after treatment with TTA or TDG tended to be higher than after treatment with their respective derivatives with triple bond in omega-2 position. The expression of ACOX, which is constitutes a target gene for PPARs, was largely unaffected by the compound treatments (FIG. 4B). Furthermore, we found no clear effects on HIF1a expression (FIG. 4C). In order to evaluate regulation of the pyruvate dehydrogenase complex (PDH), which has a key role in coordinating mitochondrial oxidation of different energetic substrates, we measured the expression of PDH kinase 1 and 4 (PDK1 and PDK4) and SIRT4 (FIGS. 4D-4F). For these three factors, which all mediates an inhibitory effect on PDH activity, a clear change in expression was only found for PDK4. The expression of PDK4 was significantly increased in cells treated with TTA or TDG, and with a moderate effect of 2-triple TTA and 2-triple TDG.

In this example we investigated the effects of a panel of modified fatty acids on cellular viability and mitochondrial physiology. The molecular modifications of the fatty acids aimed to modulate specific biological activities, especially the ability of the fatty acid to be degraded (oxidized) inside the cell. These new compounds were compared to the already well-characterized modified fatty acid TTA, and to resveratrol and AICAR, which are all known to induce mitochondrial adaptations. The main findings in this study were that replacement of the sulphur atom with a nitrogen atom in the 3-position of TTA, which results in TDG, caused similar effects on cell viability and mitochondrial function, and that introduction of a triple bond in omega-2 position of the acyl chain largely protected against the cytotoxic effects.

At high concentrations (100-200 μM), both TTA and TDG had severe effects on mitochondrial function and cell viability, but they were well tolerated at lower concentrations. These data are consistent with previous findings showing that TTA targets mitochondria and induces antiproliferative effects and apoptosis in cultured cancer cells of various origins. The mechanisms may overlap with previously reported mechanisms of fatty acids and lipotoxicity, but TTA also seems to have specific properties. These effects are thought to underlie the antitumor properties of TTA observed in several animal tumor models.

In animal models of nutrient utilization, obesity and metabolic syndrome, treatment with TTA has been found to reduce the levels of plasma lipids and increase hepatic fatty acid oxidation. The function of TTA as a pan-PPAR agonist may partly explain the biological activity of this compound, but PPAR independent effects has also been demonstrated in PPAR knockout rats. Partial mitochondrial uncoupling seems to be one of these PPAR-independent effects [18], and this is compatible with our data showing increased leak respiration in TTA-treated cells (FIG. 1D). Furthermore, only minor effects was observed with the modified short chain fatty acid ethylthio acetic acid and the branched chain valproic acid derivative 2-(ethylthio) pentanoic acid, which suggests that the effects of the long chain derivatives is mediated through interaction with specific factors, rather than being caused by unspecific toxic action of sulphur-substituted acyl-chains.

Introduction of a triple bond in the omega-2 position of the TTA and TDG structures, resulting in 2-triple TTA and 2-triple TDG, significantly reduced the harmful effects on cell viability and mitochondrial function. A triple bond in this position will supposedly prevent peroxisomal omega oxidation, but it remains unknown if this aspect contributes to the cytoprotective effects. Rather, it is possible that the improved cellular tolerance may be explained by differences in molecular geometry at the omega-terminal end of the fatty acid, changing the interactions with fatty acid handling proteins such as trans-membrane transporters (e.g. FAT/CD36) and metabolizing enzymes, and/or reduced affinity to regulatory proteins such as the PPARs. Interestingly, introduction of a triple bond in omega-1 position of TTA did not neutralize the effects on hepatic fatty acid oxidation and plasma lipids when the compound was administered to rats. In summary, these results suggest that that insertion of a triple bond near the terminal end of the acyl chain of TTA may prevent harmful cellular effects, while retaining the hypolipidemic potential in animals. The effects of TDG and 2-triple TDG have not yet been studied in vivo.

The HeLaNRF1/c4 live-cell reporter model was developed specifically to investigate physiological regulation of the NRF1-regulated transcriptional program of mitochondrial biogenesis. In this model, the reporter activity increased upon activation of AMPK, which is a central energy sensor that mediates actions to prevent energy crises, including induction of mitochondrial biogenesis through regulation of NRF1. In agreement with previous findings, the AMPK activator AICAR was found induce the reporter activity in our present studies. In addition, a strong induction was found after treatment with resveratrol, which activates AMPK and leads to mitochondrial biogenesis in various cell models. However, none of the modified fatty acids changed the NRF1-reporter activity in the HeLaNRF1/c4 cells. Hence, these compounds did not induce NRF1-regulated mitochondrial biogenesis, neither directly nor indirectly through AMPK. Since such a mechanism would be the anticipated response to energy depletion, it can be speculated that these compounds act without threatening the cellular energy status. Hence, for TTA, the presented data suggest that the previously reported induction of mitochondrial oxidation pathways in rat liver is most likely not mediated through activation of AMPK/NRF1. Our results support the hypothesis that TTA and similar compounds may act independently of AMPK, e.g. through the action of mTOR.

The expression of PGC1α and PDK4 was found to be higher after treatment with TTA and TDG, compared to 2-triple TTA and 2-triple TDG, respectively. These data correlated with the cytotoxic potential observed in living cells, and supported the findings that the triple bond near the terminal end of the acyl chain seems to attenuate some of the effects. Apparently, these effects did not involve changed expression of HIF1α. Increased expression of PGC1α and PDK4 is consistent with a metabolic change involving mitochondrial adaptation compatible with increased mitochondrial oxidation. Supporting the findings above, these effects did not seem to involve AMPK regulation, as the effects of treatment with TTA and TDG were different from what was seen after treatment with AICAR.

In summary, this study indicates TTA and TDG have similar cytotoxic effects when given to cell cultures at relatively high concentrations, and that these effects were prevented by insertion of a triple bond in omega-2 position. Combined with recent findings in rats, our findings may suggest that a triple bond near the omega-end of long-chain thio- and nitro-ether fatty acids may protect against cellular stress, without changing the hypolipidemic effects in vivo.

### Example 6—Effect of Modified Fatty Acids in MOLM-13 Human Acute Leukemia Cells

MOLM-13 suspension cells were diluted and added to 96-well plates (15.000 cells/well).

The modified fatty acids TTA, tr-TTA, TDG (TGH) and tr-TDG (tr-TGH) were complexed to bovine serum albumin (BSA), and 10-fold dilutions of the fatty acids or BSA control were added to the wells in duplicate. Two plates were run in parallel, and WST-1 was added to the cells after 48 (plate 1) and 72 hours (plate 2). Absorbance at 450 nm measured to assess viability of the cells.

TABLE 1
viability after 48 hours

| Abs450 nm | | BSA | TTA | Tr-TTA | TDG | Tr-TDG |
|---|---|---|---|---|---|---|
| 2 | mM | 0.67075 | 0.37085 | 0.21715 | 0.3163 | 0.5009 |
| 1 | mM | 0.87975 | 0.6214 | 0.51005 | 0.257 | 0.28325 |
| 0.5 | mM | 0.77035 | 0.89565 | 0.94625 | 0.3207 | 0.21765 |
| 0.25 | mM | 0.8867 | 1.0746 | 1.2206 | 0.5423 | 0.16795 |
| 0.125 | mM | 0.74835 | 0.80785 | 0.88685 | 0.47655 | 0.14525 |
| 0.0625 | mM | 0.8065 | 0.8466 | 0.94035 | 0.93075 | 0.4964 |

TABLE 2
Viability after 72 hours

| Abs450 nm | | BSA | TTA | trTTA | TGH | trTGH | TTP |
|---|---|---|---|---|---|---|---|
| 2 | mM | 0.46665 | 0.21145 | 0.18115 | 0.2359 | 0.426 | 0.2329 |
| 1 | mM | 0.7325 | 0.33855 | 0.37985 | 0.18935 | 0.366 | 0.73905 |
| 0.5 | mM | 0.6073 | 0.5284 | 0.9026 | 0.175 | 0.1964 | 0.94585 |
| 0.25 | mM | 0.56245 | 0.60465 | 1.1037 | 0.203 | 0.13845 | 1.1921 |
| 0.125 | mM | 0.4756 | 0.6408 | 0.88345 | 0.4813 | 0.13005 | 0.9466 |
| 0.0625 | mM | 0.5171 | 0.608 | 0.9898 | 0.80725 | 0.44525 | 0.9713 |
| 0.03125 | mM | 0.55285 | 0.5381 | 0.6456 | 0.4491 | 0.3769 | 0.61275 |

The results indicate that the TGH and tr-TGH are toxic to leukemic cells at a lower concentration than TTA and tr-TTA. The only difference between the compounds is the presence of Sulphur in beta-position (TTA) or nitrogen in beta-position (TGH).

### Example 7—TTA Influences Brain Gene Expression

3-thia fatty acid influenced brain gene expression when fed to rats for 50 weeks

The 3-thia fatty acid, tetradecylthioacetic acid (TTA), is a synthetic modified fatty acid, which influences the regulation of lipid metabolism, the inflammatory response and redox status. Specifically, TTA has been shown to improve mitochondrial function and increase mitochondrial proliferation. This study aimed to test whether TTA is passing the blood-brain barrier and affecting brain gene expression in a long-term experiment (50 weeks of feeding).

This animal study was conducted according to the Guidelines for the Care and Use of Experimental Animals, and the protocol was approved by the Norwegian State Board of Biological Experiments with Living Animals. 8 to 10-weeks old male Wistar rats, weighing 200-250 g, were obtained from Taconic Europe A/S (Denmark). After one week acclimatization, they were divided in groups of 8 animals and fed a control, high fat (25% w/v) diet or a high fat diet supplemented with either TTA (0.375% w/v) or FO (10% w/v), or their combination. After 50 weeks, the rats were anaesthetized with Isofluorane (Forane, from Abbot Laboratories Ltd, Illinois, USA) inhalation under non-fasting conditions, and the brains were immediately removed, frozen in liquid nitrogen, and stored at −80° C.

Total cellular RNA was purified from 100 mg brain tissue using RNeasy Mini Kit (Qiagen). RNA was quantified spectrophotometrically (NanoDrop 1000, NanoDrop Technologies, Boston, Mass. USA), and the quality was evaluated by capillary electrophoresis (Agilent 2100 Bioanalyzer, Agilent Technologies, Palo Alto, Calif., USA). For each sample, 1 ug total RNA was reversely transcribed in 20 □l reactions using Applied Biosystem's High Capacity cDNA Reverse Transcription Kit with RNase inhibitor according to the manufacturer's description. Real-time PCR was performed on 384-well microfluidic plates with custom-made probes and primers from Applied Biosystems [Foster City, Calif., USA]. The genes selected were: Cpt1a, Cpt2, and Ucp3. Two different control genes were included: 18s [Kit-FAM-TAMRA (Reference RT-CKFT-18s)] from Eurogentec, Belgium, and Arbp from Applied Biosystems. In comparative analysis using the programs Normfinder, Arbp was found to be the best. The expression value of each gene in each sample was normalized against this endogenous control.

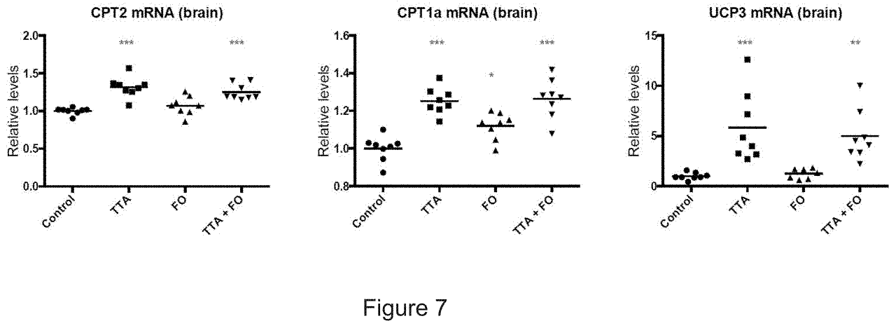

Brain gene expression of carnitine palmitoyl transferase 2 (Cpt2), a PPARα response gene involved in the import of fatty acids to the mitochondria for β-oxidation, was increased by TTA (FIG. 7). Cpt1a, also involved in mitochondrial fatty acid import, was increased by both TTA and fish oil, but no additive effects were observed in the combined treatment group. In addition, uncoupling protein 3 (Ucp3), involved in mild uncoupling in mitochondria, often induced during high mitochondrial activity, was increased by TTA.

Brain gene expression of genes involved in mitochondrial function was influenced in rats fed TTA-diets for 50 weeks, demonstrating that TTA is able to cross the rat blood-brain barrier and influence mitochondrial function in brain.

### Example 8—TTA Prevents Mildronate-Induced Fatty Liver

### 3-Thia Fatty Acid Prevents Mildronate-Induced Fatty Liver in C57BL/6 Mice.

Carnitine depleted, 3-(2,2,2-trimethylhydrazinium)propionate (Mildronate: meldonium) treated C57Bl/6 mice will develop fatty liver due to inhibition of mitochondrial β-oxidation. We studied whether tetradecylthioacetic acid (TTA), a mitochondrial targeted compound, could alleviate Mildronate-induced fatty liver.

C57BL/6 mice were divided in 4 groups of 10 mice and fed a control low-fat diet, low-fat diets with TTA (720 mg/kg body weight), Mildronate (550 mg/kg body weight) or a combination of these two treatments for 3 weeks (n=10). At sacrifice, liver samples were collected, lipids were extracted, and triacylglycerol (TAG) measured on the Hitachi 917 system (Roche Diagnostics, GmbH, Mannheim, Germany) using a kit from Roche Diagnostics.

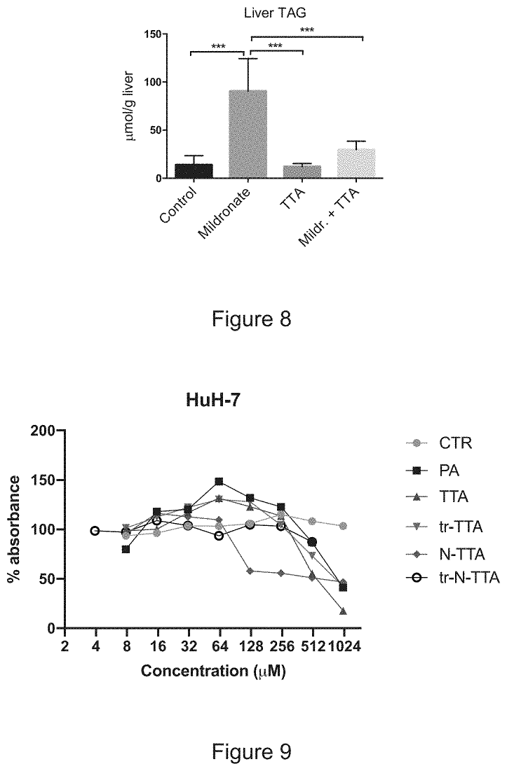

Mildronate treatment in mice resulted in fatty liver as demonstrated by a 7-fold increase in the main liver lipid class, TAG. Strikingly, TAG was significantly reduced by TTA and Mildronate co-treatment compared to Mildronate treatment alone (FIG. 8). This shows that TTA was able to prevent Mildronate-induced fatty liver.

### Example 9—Effect of Various Modified Fatty Acids on Neurodegenerative Diseases (ND)

The fatty acids (FAs) TTA, tr-TTA, N-TTA, tr-N-TTA and (PA) were complexed with FBS following the same procedure, differing only in final stock concentration, due to variation in solubility. Approximately 0.07 g of FA was weighed in a 10 ml sterile serum tube (BD Vacutainer®, catalog no. 368430) and dissolved in sterile filtrated 0.1 M sodium hydroxide (NaOH) of a volume giving 25 mM FA, controls without FA were made, containing only NaOH and FBS. A syringe (Sterican®, catalog no. 4657577) was pierced through the rubber cap of the serum tube, and the tube was placed in an 80° C. water bath until the FAs were dissolved. Each FA-solution was then diluted in inactivated FBS to stock concentration 1 mM (N-TTA, tr-N-TTA), and 2 mM by adding the FA to a volume of 100% FBS after heating the FBS to 45° C. in a water bath. The transfer of FA to FBS had to be executed controlled and swiftly to avoid the formation of precipitates. If any precipitates in the solution were visible, the FA and a new volume of FBS were reheated, and the procedure performed again. Following successful complexation, the stock solutions were stored at −20° C. in 1 ml aliquots (CryoPure tube, Sarstedt AG & Co, Nümbrecht, Germany).

Selected FAs (TTA, tr-TTA, PA) were weighed in and dissolved in 100% DMSO yielding stock concentrations of 50 mM (table 4.1.). The stock solutions were stored at −20° C. in 1 ml aliquots (CryoPure tube, Sarstedt AG & Co, Nümbrecht, Germany).

### HuH-7 Cell Line

The HuH-7 cell line (JCRB0403) was taken over in passage 35 and experiments were performed up to passage 44. The cell line was used to have a familiar cell line with positive results of the treatments in earlier experiments and served as a reference. It was also used to see if similar results as achieved in the aforementioned experiments could be reproduced to establish successful preparation of the FAs.

The cells in the long-time storage solution were thawed to room temperature and resuspended by pipetting the solution up and down. 1 ml of the cells in solution were then added to approximately 9 ml of pre-heated HuH-7 cell medium (DMEM High Glucose, 10% FBS and 1% PenStrep, see table 4.2.) at 37° C. in a 25 cm²-flask (Falcon®, Corning Ink., NY, USA). The cells were then placed in an incubator with 5% CO₂ at 37° C. After 24 hours, the cell medium was changed with a new volume of 10 ml HuH-7 cell-medium to remove the residual DMSO from the freezing medium.

During cultivation, half of the cell medium was aspirated after 2-3 days and replaced with the same volume of new cell medium pre-heated to 37° C. Generally, all components added to the cells were pre-heated to 37° C. in a water bath unless something else is described.

The cells were passaged when they had reached approximately 70-90% confluency. Following aspiration of the cell medium, 3 ml PBS was added to the culture vessel, and the flask was carefully rotated to rinse the cells. After aspiration of the PBS, 2 ml trypsin was added to the cells, and the flask was carefully rotated and placed in the incubator (37° C., 5% CO₂) for 5 minutes. The flask was then quickly checked for cell detachment under the microscope (Nikon TMS Inverted Phase Contrast Microscope, Tokyo, Japan), and 3 ml cell medium was added to the cells to deactivate the trypsin. To further loosen the cells from the flask-surface, the bottom of the flask was gently tapped, and if necessary, a pipette was used to flush the cells from the surface. The cell suspension was then transferred to a 15 ml centrifugation tube for a complete settlement. Centrifugation was performed in room temperature for 3 minutes at 200 g. Then, the supernatant was carefully aspirated off, and the cells were resuspended in a fixed volume cell medium, usually 5 ml. To continue the cell line, the cells in suspension were usually split between 1:10 and 1:15 depending on the confluency before passaging. A 1:10-split means transferring 0.5 ml of 5 ml resuspended cells to a new 25 cm² flask for cultivation.

Prior to cell viability assays and cell treatments, the cells needed to be counted to ensure an appropriate seeding density. Cells were obtained from the resuspended solution following the procedure from cell passaging. 10 μl Trypan blue was mixed with 10 μl cell suspension and partitioned to both chambers of the Countess® Cell Counting Chamber Slide (Gibco® Invitrogen, Paisley, Scotland, UK). The cells were counted using the Countess II Automated Cell Counter (Invitrogen, Carlsbad, Calif., USA), and the mean value between the two measurements was used.

### SH-SY5Y Cell Line

The SH-SY5Y cell line (ECACC 94030304) was taken over in passage 14 and were thawed and cultivated following the same procedure as for the HuH-7 cell line, but with some modifications. The cell medium was a 1:1 mixture of DMEM and F-12 Ham, and it was added 1 NEAA, 1% 2 mM L-glutamine, 15% FBS and 5% PenStrep to the growth medium (table 4.2.).

During cell passaging, 1 ml trypsin was used, and the flask was placed in the incubator for 1 minute. The cells were split when the cells were approximately 80% confluent.

WST-1 assays were performed up to cell passage 28. The 4-days treatments prior to gene analysis were in passage 30, whereas the 1-day treatment was in passage 16.

### Cell Viability Assay WST-1

The WST-1 cell proliferation assay was performed using the kit from Roche (table 4.2.) and its accompanying procedure (version 16, February 2011) (134). The number of cells seeded to each well in the 96-well plates (Corning Inc., catalog no. 353072) varied depending on the treatment time and cell type (table 3.).

TABLE 3
Cell type and seeding densities for the WST-1 assay

| | | Seeding density |
|---|---|---|
| Cell Type | Days of Treatment | (cells/well) |
| HuH-7 | 2 | 9000 |
| HuH-7 | 4 | 7000 |
| SH-SY5Y | 4 | 9000 |

Cell suspension and medium were then transferred to each well in a volume of 100 μl in total. Subsequently, the plates were incubated for 24 hours to make the cells settle. The next day, 100 μl with FA complexed with FBS or diluted in DMSO was added to row A or B in the 96-well plate to give a defined concentration. The solution was then mixed with a pipette, and 100 μl of the medium with FA was transferred to the next well in the same column, mixed by pipetting up and down at least five times before 100 μl was transferred again to the next well in the column. This procedure was repeated until the second last, or last well in each column was reached, making a series of two-fold dilution of the FAs in the cell medium. Two parallels were performed for every treatment per run. After the addition of FAs, the plates were incubated for 24-96 hours depending on the length of the treatment.

Following the FA-treatment, the cells were studied under a microscope to ensure that visual observations matched the measured absorbance and cell activity in the assay. Subsequently, 10 μl WST-1 reagent was added to each well, and the plates were incubated for 3 hours. Due to high activity in the HuH-7 cell line, the WST-1 reagent was diluted 1:1 with PBS. Before the measurement of absorbance, the plates were placed on a shaker for 1 minute ensuring thoroughly mixing of the wells. The plates were then put straight into the spectrophotometer (SpectraMax Plus 384 Microplate Reader, Molecular Devices Corporation, San Jose, Calif., USA) to perform the measurements. The accompanying software was used (SoftMax Pro, Molecular Devices Corporation, San Jose, Calif., USA) to obtain the results. The absorbance of the samples was measured at 450 nm, and the reference wavelength was set to 650 nm. For each well, the measured absorption at 650 nm was subtracted from the absorption at 450 nm. The measured absorbance for the treated cells was then compared to the controls (blank) containing only cells and growth medium.

TABLE 4
Example of layout for the WST-1 assay in 96-well plates, 4 days
of treatment. The measurements were performed in duplicates using
two columns for each dilution series. The outer wells were filled
with PBS to minimize loss of fluids due to evaporation.

| | 1 | 2-3 | | 4-5 | | 6-7 | | 8-9 | | 10-11 | 12 |
|---|---|---|---|---|---|---|---|---|---|---|---|
| A | | | | | | | | | | | |
| B | | CTR | | PA | | TTA | | tr-TTA | | Blank | |
| | | 1 | mM | 1 | mM | 1 | mM | 1 | mM | | |
| C | | 0.5 | mM | 0.5 | mM | 0.5 | mM | 0.5 | mM | Blank | |
| D | | 250 | μM | 250 | μM | 250 | μM | 250 | μM | Blank | |
| E | | 125 | μM | 125 | μM | 125 | μM | 125 | μM | Blank | |
| F | | 62.5 | μM | 62.5 | μM | 62.5 | μM | 62.5 | μM | Blank | |
| G | | 31 | μM | 31 | μM | 31 | μM | 31 | μM | Blank | |
| H | | | | | | | | | | | |

Cell Treatment with Fatty Acids

Cells from both cell lines were obtained and counted as previously mentioned. After cell counting, the cells were seeded to 6-well plates (Corning Inc., catalog no. 353046) with a density dependent on total treatment time and cell line. The cells were seeded in 3-4 ml medium and placed in the incubator for approximately 24 hours for the cells to settle. Following the 24 hours, the FAs were thawed in room temperature, the cell medium was aspirated off, and a new, exact volume was added to each well, depending on the final concentration of FA or control. Before the addition of FAs, the FA-solutions were mixed by pipetting up and down several times. The FAs were then added with an exact volume to the wells giving a total volume of 3 ml or 4 ml depending on treatment time, yielding the target concentration of FA in well. After the addition of FA to one well, the solution was mixed by carefully rotating the 6-well plate. The same procedure was followed for all the wells, differing only in FA or control added, and the ratio of medium and FA depending on treatment dose. During cell treatment, the plates were incubated under the same conditions as previously mentioned. PBS was placed between the wells to reduce liquid-loss due to evaporation. After treatment and incubation for 24-96 hours, the procedure of RNA purification was followed.

### Gas Chromatography—Mass Spectroscopy

The GC-MS analysis was performed on treated SH-SY5Y cells and its medium prior and after treatment of TTA complexed with FBS. The technical work and analysis were performed by Pavol Bohov.

Total medium and cellular FA composition were analyzed by gas-liquid after preparation of FA methyl esters from total lipids by heating with a 1:1 mixture of methanol and toluene at 90° C. for 1 hour using sulfuric acid as a catalyst. Gas chromatograph Trace GC Ultra (Finnigan, USA) was equipped with a programmable temperature vaporization injector, flame-ionization detector, AS 2000 autosampler and with a fused silica capillary column coated with dimethylpolysiloxane stationary phase (J & W Scientific, USA). Hydrogen was used as a carrier gas. The column temperature was programmed from 110° C. to 310° C. with a gradient of 2.5° C./min.

The GC signal was acquired and evaluated with Chromeleon software version 6.80 (Dionex, USA). Peaks were identified by means of known FA standards and by means of mass spectra, obtained by GC-MS analysis (GCQ MS Detector, Finnigan, USA) on the same column.

The quantification of the FAs was based on the areas of the chromatographic peaks which were measured electronically. Subsequently, the areas were converted to weight units based on the known amount of the internal standard C21:0 which had been added to the sample. The response of the analytical procedure was determined with a mixture of pure standards (Sigma-Aldrich, USA; Supelco, USA; Larodan Fine Chemicals, Sweden).

### A 12-Week Study of ApoE^{-/-} Mice Fed a TTA Supplemented Diet

The animal study was conducted according to the national (D.L. 116, G.U. Suppl. 40, Feb. 18, 1992, Circolare No. 8, G.U. July 1994)) and international laws and policies (EEC Council Directive 86/609, OJL 358, 1, Dec. 12, 1987: Guide for the Care and Use of Laboratory Animals, United States National Research Council, 1996). The Italian Ministry of Health approved the protocol (115). Twelve mice in the control group were fed a high-fat diet (23.7% w/w) and twelve mice in the intervention group were fed the high-fat diet supplemented with 0.4% w/w TTA. In addition, and not published, mice in a third group were fed the high-fat diet supplemented with 0.75% w/w TTA. After 84 days of treatment, the mice were anesthetized with 2% isoflurane and blood was removed by perfusion with PBS. Organs, including brain samples from 6 different animals in each of the three groups, were harvested, snap-frozen and stored at −80° C. until further processing. Samples of different brain regions were isolated, including the pre-frontal cortex, brain stem, hippocampus and samples termed “rest of the brain”. In this project, only samples from the latter were used.

### A 50-Week Study of Wistar Rats Fed a TTA Supplemented Diet

The main focus of the original study was to investigate the long-term effects in vivo of TTA and fish-oil, added separately or in combination, to the diet of Wistar rats (110). This included quantification of lipids in liver and plasma, measuring enzyme activities in liver, gene expression analysis in liver, and investigation of oxidative damage in liver and mitochondria.

The animal study was conducted according to the Guidelines for the Care and Use of Experimental Animals, and the protocol was approved by the Norwegian State Board of Biological Experiments with Living Animals. The two groups of interest in this project where the control group of rats fed a high-fat diet (25%) and the intervention group fed the high-fat diet supplemented with 0.375% TTA. Organs, including brains from some of the animals, were harvested after the rats were anesthetized with 2% isoflurane inhalation under non-fasting conditions and the blood had been drawn by cardiac puncture. The brains were frozen whole, not freeze-clamped, at −80° C. prior to further analysis.

### Gene Expression

RNA-purification and gene expression analysis were performed on the treated cell lines and the brain samples from the apoE^{-/-} mice as described further down. The RNA-purification of the brain samples from the Wistar rats was carried out before this project using the same procedure and protocol as for the apoE^{-/-} mice. The subsequent cDNA synthesis was performed in the same way as described here, but with 1 μg mRNA in every 20 μl reaction. Kari Williams performed the subsequent gene expression analysis. Brain samples from 8 randomly selected animals in each group of rats were analyzed.

### Total RNA Purification

The first steps in the RNA purification protocol differ between the treated cells and the brain tissue from TTA-fed mice and will be described separately.

### RNA Purification Protocol: HuH-7- and SH-SY5Y Cells

Purification of total RNA from treated HuH-7- and SH-SY5Y cells was done following the protocol “Purification of Total RNA from Animal Cells using Spin Technology” described in the RNeasy Mini Handbook (fourth edition, June 2012) with some modifications. The RNeasy Mini Kit was used.

RNA Purification Protocol: Brain Tissue from ApoE^{-/-} Mice

Purification of total RNA from brain tissue retrieved from the study described in 4.X.1 was performed using the RNeasy Lipid Tissue Mini Kit, following the procedure described in the RNeasy Lipid Tissue Mini Handbook with minor modifications. Before starting the procedure, one 5 mm stainless-steel bead (Qiagen, catalog no. 69929) was added to each 2 ml microcentrifuge tube using the TissueLyser Single Bead Dispenser (Qiagen, catalog no. 69965) and placed at −80° C. overnight. Also, approximately 30 mg of brain tissue had been weighed in on beforehand to determine the amount of tissue necessary when executing the protocol.

### RNA Quality and Quantity

The RNA concentration and quality of the samples were measured using the QIAxpert (Qiagen, Hilden, Germany) spectrometer and following the protocol in the QIAxpert User Manual. During the procedure, the samples were stored on ice. After mixing to ensure homogeneity, 2 μl from each sample was added to the slides (QIAxpert Slide-40, catalog no. 990700) including a water sample as blank. When all the samples were loaded, the slide was immediately injected to the machine for measurement, and the instructions on the screen were followed. The measurements were executed using the A260 RNA application. The measured concentrations and quality values were stored, and the RNA samples were placed at −80° C. for storage or diluted with RNase-free water to equal concentrations for further use. For the cell lines and the apoE^{-/-} mice, the RNA-integrity was not tested.

To measure and ensure RNA quantity and quality from the purified RNA from the Wistar rats, the method differed from the one used in the other samples. The RNA was quantified spectrophotometrically (NanoDrop 1000, NanoDrop Technologies, Boston, Mass. USA), and the quality was evaluated by capillary electrophoresis (Agilent 2100 Bioanalyzer, Agilent Technologies, Palo Alto, Calif., USA).

cDNA Synthesis Using Reverse Transcription Reaction

Frozen RNA samples were thawed on ice or used directly after the RNA purification protocols. All samples from the cell lines were diluted with nuclease-free water to 50 ng/μl, and all samples from brain tissue were diluted to 200 ng/μl prior to cDNA synthesis. UHRR and UMRR were initially at 50 ng/μ1 (table 4.3.). The reactants (table 4.6.) used in the RT-mix were thawed on ice and mixed following the protocol “High Capacity cDNA Reverse Transcription Kits” (Applied Biosystems, June 2010) (153). From the master mix containing the total volume of the mixed reactants, 10 μl was added to each sample tube placed on ice already containing 10 μl of the diluted RNA. The samples with a total volume of 20 μl were mixed by pipetting up and down several times. In addition, samples without reverse transcriptase (−RT) were made, using nuclease-free water to replace the RT volume. The tubes were placed on ice until they were loaded to the GeneAmp* PCR system 2700 (Applied Biosystems, Foster City, Calif., USA) and the program described in table 4.7. was used. After the procedure, all samples were diluted to 10 ng/μl by the addition of nuclease-free water and placed for storage at 4° C. until further use.

### Real-Time Quantitative Polymerase Chain Reaction

Real-time qPCR was used in this project to investigate the possible effects of TTA and similar modified FAs on gene expression. The cDNA synthesized by RT were vortexed and diluted based on test runs and expected expression of the respective genes, to give an acceptable Ct-value within the limit of the experiments.

The mix for the samples (and standard curves) was made using TaqMan Gene Expression Master Mix (table 4.8.), probes specific for the genes in question, and nuclease-free water. −RT and NTC samples were always used in the experiments. The master mix for each gene was made according to table 4.8. and added to tubes together with the cDNA. The tubes were vortexed and centrifuged briefly before the samples were added to a 384-well plate (Sarstedt AG & CO, catalog no. 72.1984.202) with the use of a pipette. For every sample, three parallels of 9 μl were used. After the addition of all the samples to the plate, a plastic cover (Sarsted AG & CO, catalog no. 95.1994) was used to seal the plate before centrifuging it for 2 minutes at 700 g. The prepared samples were placed in the PCR cycler (ABI PRISM 7900HT Sequence Detection System, Applied Biosystems) for quantification, using the 9600 Emulation Thermal Cycler Protocol (table 4.9.) in the software SDS 2.3 (Applied Biosystems, Foster City, Calif., USA).

When performing the qPCR reactions, all samples were analyzed in triplicates, and the mean value was used in further analysis. The 2^{-ΔΔCt} method was used to analyze the qPCR results for the treated cell lines and the apoE^{-/-} mice. Relative gene expression compared to reference genes in the Wistar rats was done using generated standard curves. The latter method was also tested for the apoE^{-/-} mice to compare and see if it gave any differences in results.

### Statistics

Microsoft® Excel 2019 (Microsoft Corporation, Redmond, Wash., USA) for Mac was used for handling of data and initial calculations of results, whereas GraphPad Prism 8 (GraphPad Software Inc., San Diego, Calif., USA) was used for statistical analyses and generation of figures and graphs. Results are shown as mean values with their standard deviation when possible. The determination of significant statistical differences between groups was done by using the one-way analysis of variance (ANOVA) and the Dunnett's multiple comparisons test. In addition, the Student's t-test (unpaired, two-tailed) was performed when relevant. P-values ≤0.05 were considered statistically significant and denoted with *. Furthermore, P-values ≤0.01 were denoted **, P-values ≤0.001 denoted *** and P-values ≤0.0001 denoted ****.

### Results

### Cell Viability Assay WST-1

Treatment with FAs complexed with FBS over 2 days was performed once for the HuH-7 cells. The absorbance was measured every hour for five hours after the addition of WST-1. The results after three hours can be seen in FIG. 9. The untreated cells had a mean absorbance value at approximately 1.5 and were used as a reference (100%). The control containing only FBS and NaOH had similar absorbance and therefore activity at all concentrations compared to the untreated cells. PA had increased activity at concentrations between 16 μM and 250 μM, going up to almost 150% at 62.5 μM compared to the untreated cells. TTA did also increase activity between the same concentrations as PA, but with a slightly lower top at 62.5 μM with about 130% activity compared to untreated cells. The same could be observed for tr-TTA, with increased activity from the lowest concentration, 8 μM, to 250 μM with the same top at 62.5 μM on 130% as TTA. N-TTA stimulated activity between 16 μM and 62.5 μM with a top at 16 μM where the activity was measured to 116% compared to untreated cells. The treatment with tr-N-TTA measured activity around 100% for all concentrations with a top at 16 μM on 109%. For most treatments (PA, TTA, tr-TTA, N-TTA), a decrease in activity was detected at 500 μM and 1000 μM with activities ranging between 20-75% compared to no treatment, whereas tr-N-TTA at 500 μM had activity at almost 90%. N-TTA did also have a decreased activity at 125-250 μM between 50-60%.

Overall, TTA and tr-TTA had similar effects on cell activity as PA based on the WST-1 assay. Observations of the cells under the microscope before the addition of WST-1 corresponded to the measured absorbance.

FIG. 9 shows the results of WST-1 assay after 2 days of treatment with different FAs and controls in a 2-fold dilution series.

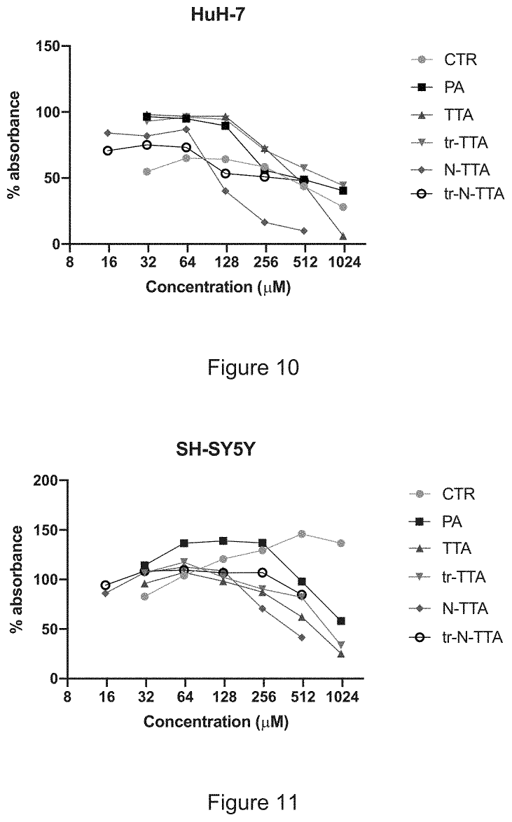

Treatment with FAs complexed with FBS over 4 days was performed two times for the HuH-7 cells. The measured absorbance three hours after the addition of the WST-1 reagent can be seen in FIG. 10. The untreated cells had a mean value of absorbance of 2.6 and is set to 100% absorbance in the figure. Contrary to the 2-days treatment, the CTR had lower activity than the untreated cells for all concentrations with activities ranging between 30-65%. For the PA-, TTA, and tr-TTA-treatments, the activity was similar to the untreated cells up to 125 μM from where it started to decrease. For N-TTA and tr-N-TTA, the activity was at its highest and stable up to 62.5 μM, from where it decreased to between 10-50% at higher concentrations. TTA had almost no activity at 1000 μM, indicating cell death. The same result was true for N-TTA.

Observations of the cells under the microscope before the addition of WST-1 corresponded to the measured absorbance.

FIG. 10 shows the results of WST-1 assay after 4 days of treatment with FAs and controls in a 2-fold dilution series.

Treatment with FAs complexed with FBS over four days was performed four times for the SH-SY5Y cell line. The measured absorbance three hours after the addition of the WST-1 reagent can be seen in FIG. 11. Generally, the activity in the SH-SY5Y cells was lower than for the HuH-7 cells. The mean value of absorbance for the untreated cells was at about 0.6. The CTR-treatment had absorbance levels at about 120-130% compared to the untreated cells. The activity was at 80% in the lowest concentration and approximately the same for the second lowest concentration. The activity then increased with increasing level of FBS in the medium. PA stimulated activity in concentrations up to 250 μM and was the same as the control when at 500 μM. TTA only stimulated activity at 62.5 μM (107%), but it was approximately the same as in no treatment for 125 μM. tr-TTA induced activity up to 125 μM (117%) and N-TTA between 31-125 μM at about 110%. The last FA, tr-N-TTA, had activities around a 100% for all concentrations except at 500 μM where it was slightly lower than control (84%). All treatments had decreased activity towards the highest concentration.

Observations of the cells under the microscope corresponded to the measured absorbance to some degree, but it was less clear than in the observations of the HuH-7-cells.

### Gas Chromatography—Mass Spectrometry

The GC-MS analyzing the amount of TTA in cell medium and inside the SH-SY5Y cells was conducted as described. The obtained results are presented in table 5.

TABLE 5
Results of GC-MS analysis of total FA and TTA in cell medium and SH-SY5Y. Weight
percent (wt %) of total FA in parenthesis. Coefficients of variation were
far below 5%, and it could be concluded that the results were accurate.

| | | | |
|---|---|---|---|
| Compound | Medium start | Medium 24 h | Cells 24 h |

| | | | | | | |
|---|---|---|---|---|---|---|
| TTA | 124 μM | (39.7%) | 112 μM | (37.5%) | 1.71 μM | (18.7%) |

| | | | | | |
|---|---|---|---|---|---|
| TTA: 1n-8 | — | 0.53 μM | (0.18%) | 0.017 μM | (0.19%) |

| | | | | | | |
|---|---|---|---|---|---|---|
| Total TTA | 124 μM | (39.7%) | 113 μM | (38.4%) | 1.73 μM | (18.9%) |
| Total FA | 312 μM | (100%) | 298 μM | (100%) | 9.157 μM | (100%) |

As table 5 demonstrates, the initial TTA concentration in the medium just after the addition was measured to be 124 μM, whereas the target concentration was 125 μM. The total amount of TTA accounted for 40% of the total FAs in the medium. After incubation for 24 hours, the total TTA concentration went down to 112 μM in the medium. One of TTA's metabolites, TTA:1n-8, could also be detected in the cell medium. The TTA concentration inside the SH-SY5Y cells after 24 hours was found to be 1.71 μM, accounting for approximately 19% of the total FA amount in the cell.

### Gene Expression Analysis

The qPCR efficiency for the selected genes in the project was within the limits of 90-110% based on the slopes of the generated standard curves. Small differences in Ct-values between the triplicates of each sample was also a good indication for high efficiency and repeatability. The use of −RTs indicated efficient elimination of DNA during RNA purification in all experiments, and the NTCs gave no manifestations of contamination throughout the process prior to the gene expression analyses.

The reproducibility of the qPCR assay can be controlled by comparing the Ct-values of the triplicated samples. Ideally, it should be less than 0.5 (138). In all experiments, the difference in Ct-values between triplicates was usually less than 0.2.

### Gene Expression Analysis—Cell Lines

Based on the WST-1 results, doses of 62.5 μM and 125 μM of FAs complexed with FBS were chosen for the treatment of the cell lines.

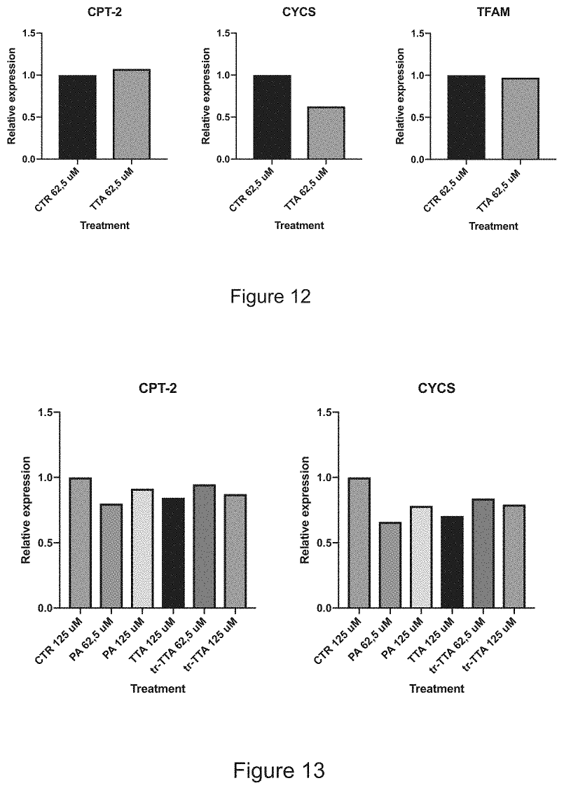

Gene expression analysis was first performed on treated SH-SY5Y cells, and the results can be seen in FIG. 12. The genes analyzed were CPT-2, CYCS and TFAM. One parallel of each treatment was analyzed using the 2^{-ΔΔCt} method with HPRT1 as the reference gene. CPT-2, which regenerates fatty acyl-CoA after FA transport across the mitochondrial and activates it for subsequent β-oxidation, did not show any signs of an altered gene expression when comparing the control with 62.5 μM TTA treatment. The same result applied for TFAM; whose function is to participate in the replication of mtDNA. CYCS, encoding for the electron carrier cytochrome c functioning in the ETC, revealed a lower relative expression in the TTA-treated SH-SY5Y cells compared to control.

Analyses of additional treatments of SH-SY5Y can be observed in FIG. 13. As for the first experiment, no tendency in altered gene expression was observed compared to the control of the selected PPAR target genes. If anything, all treatments had a slightly lower or similar expression of CPT-2. A small decrease in the CYCS gene expression was seen compared to the FBS-control, particularly for TTA and PA.

Lack of signs of PPAR activation in the initial experiments treating the SH-SY5Y cells was followed by treatment of HuH-7 cells to see if any changes in relative gene expression could be observed in that cell line.

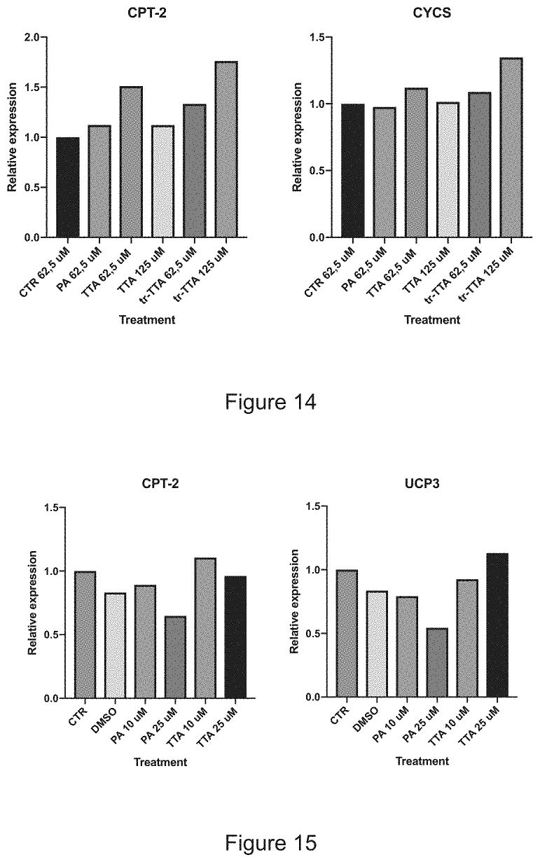

One parallel of each treatment was analyzed using the 2^{-ΔΔCt} method with 18S as a reference gene, and the results can be seen in FIG. 14. Relative expression of CPT-2 was about 1.5 times higher for the treatment of TTA 62.5 μM relative to the control at 62.5 μM. Similarly, the relative expression of CPT-2 in the treatment with tr-TTA 125 μM was about 1.7 times higher relative to the control at 62.5 μM. Compared to the TTA 62.5 μM, the treatment with 125 μM TTA gave no increase in relative expression compared to control. The treatments did not affect the relative expression of CYCS except for tr-TTA at 125 μM where it was 1.3 times higher than in the 62.5 μM control.

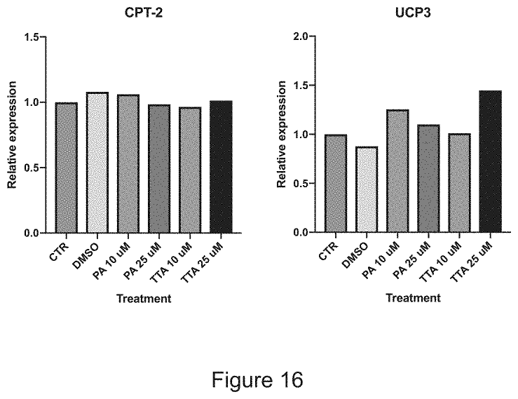

The aforementioned treatments were performed using FAs complexed with FBS. Due to relatively small changes, if anything, in gene expression for these treatments, FAs dissolved in DMSO were tried. Precipitations were observed for all FAs in DMSO when added to the cell medium in final concentrations above 25 μM, and further analysis was only performed for low concentrations of FAs. A control with the highest concentration of DMSO achieved in the FA solutions was also included for comparison in both cell lines. Effects of treatment with FAs dissolved in DMSO on the HuH-7 cells and the SH-SY5Y cells can be seen in FIGS. 15 and 16 respectively. The duration of treatment was 4 days.

No effect on changes in relative gene expression for CPT-2 could be seen in any of the treatments of the HuH-7 cells compared to control. If anything, the CPT-2 expression for PA at 25 μM was lower with a relative expression of 0.65 compared to control. UCP3 encodes for one of the UCP homologs involved in mitochondrial uncoupling of ETC and ATP synthesis. As for CPT-2, no effects on gene expression was observed for UCP3 except for a decreased relative expression in the 25 μM PA-treatment at approximately 0.5 compared to untreated cells.

FIG. 15 shows the effects of treatment with FAs dissolved in DMSO in HuH-7 cells after 4 days of treatment.

Changes in gene expression for the selected genes in the SH-SY5Y cells were also absent, except from a slightly elevated level of expression at about 1.5 times as high of UCP3 relative to control (FIG. 16).

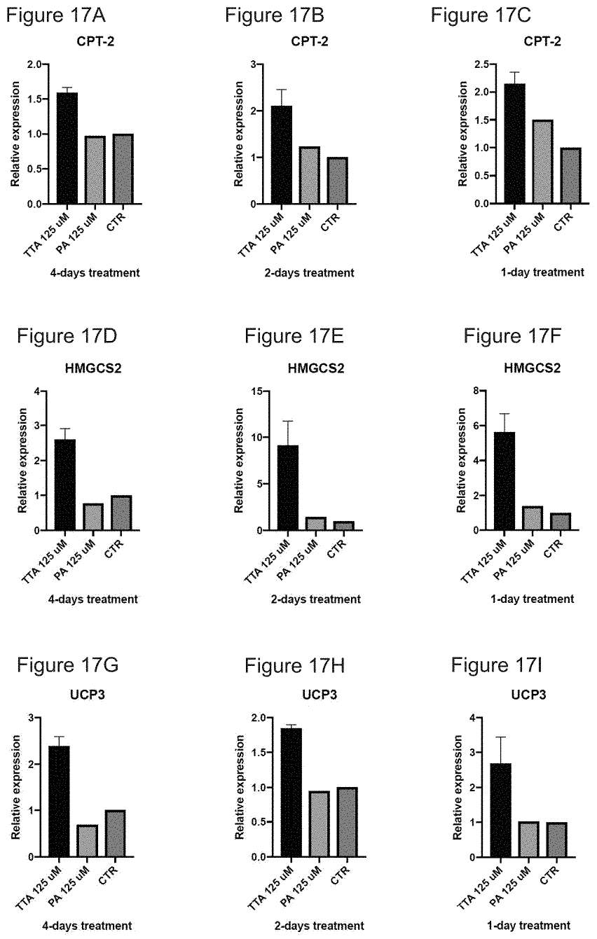

Due to effects not being as expected in the experiments, analyses of gene expression in HuH-7 cells were performed on an expanded selection of PPAR target genes. This included the HMGCS2 gene encoding for a protein catalyzing the first step in the pathway converting acetyl-CoA to KBs. Two parallels of TTA 125 μM complexed with FBS were compared to one parallel of PA 125 μM and one parallel of CTR (FBS/NaOH). To see if there was any effect of treatment time, all treatments were performed over 1, 2 and 4 days. Gene analyses were performed on three selected PPAR target genes, CPT-2, HMGCS2, and UCP3, and the results can be seen in FIGS. 17A-17I.

Treatment with 125 μM TTA gave a 1.5-fold increase in CPT-2 expression after 4 days, whereas the 2-days and 1-day treatment gave a 2-fold increase relative to control. 125 μM PA did not result in increased expression in the 4- and 2-days treatment, but a 1.5-fold increase for the 1-day treatment in CPT-2. The relative expression of HMGCS2 after treatment with PA 125 μM was between 0.8 and 1.4 depending on the treatment period. The 4-days treatment of TTA gave approximately 2.5-fold increase of HMGCS2 relative to control. The 2-days treatment of TTA yielded a mean value at about 10-fold increased expression of HMGCS2. Similar results were obtained after 1-day treatment with a 5-6-fold increase in HMGCS2 relative to control for the TTA-treatment. The relative expression of UCP3 had a 2-3-fold increase in all treatment periods with for TTA. The PA-treatments did not induce any changes in gene expression relative to control beyond what is already mentioned.

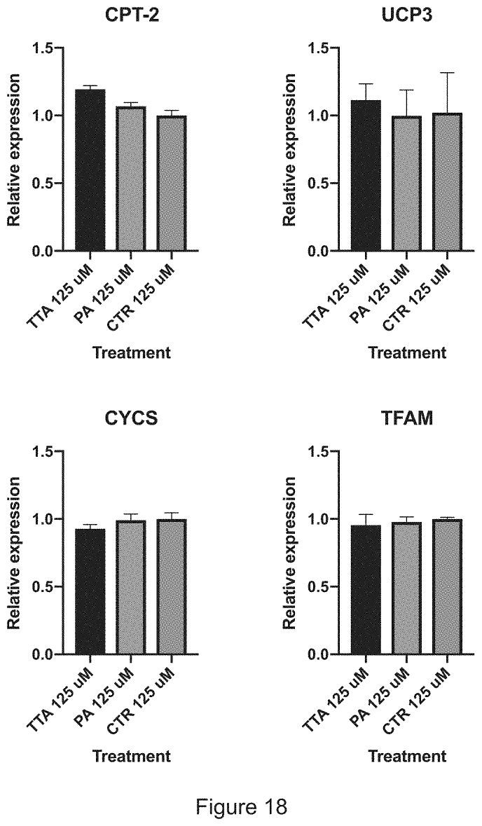

A last experiment on the SH-SY5Y cell line was performed with TTA complexed with FBS at 125 μM and a treatment period of 24 hours. The results of the relative expression of selected genes can be seen in FIG. 18. Two parallels of each treatment with TTA, PA, and CTR were performed. No change in relative gene expression compared to control could be seen for CPT-2, UCP3, CYCS, and TFAM. Analysis of HMGCS2 and FGF21 was also performed, but the expression of the genes was not detected. FGF21 is stimulated by HMGCS2-activity and serves to, among other things, stimulate glucose uptake in hepatocytes.

### Gene Expression Analysis—ApoE^{-/-} Mice

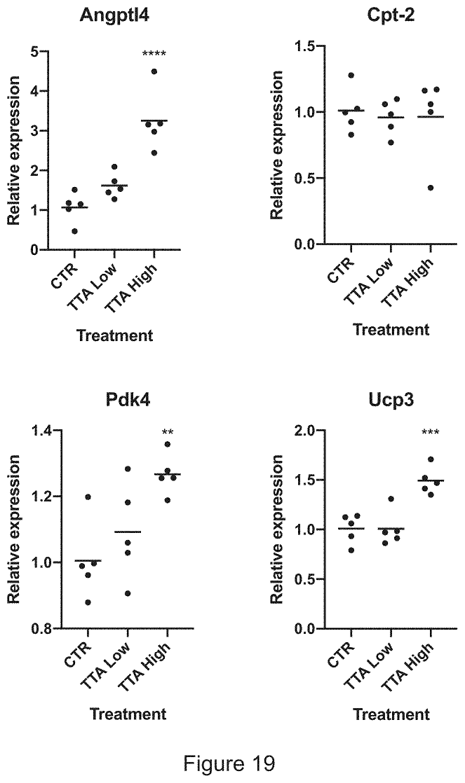

Gene expression analysis was performed on brain samples harvested from apoE^{-/-} mice as previously described, and the results can be seen in FIG. 19. The relative gene expression was calculated using the 2^{-ΔΔCt} method with Hprt1 as a reference gene (shown). For comparison, the relative gene expression using a standard curve was also analyzed showing no significant differences compared to the 2^{-ΔΔCt} method (not shown). To see if the choice of reference gene could affect the results, the 2^{-ΔΔCt} method was also used with 18S as a reference gene. Here too, no difference in results were observed (not shown).

The results from the gene expression analysis did not show any significant differences in mice fed the low-TTA diet compared to control. This was also true for the relative expression of Cpt-2 in mice fed the high-TTA diet.

Mice fed the high-TTA diet displayed an approximately 3-fold increased expression of Angptl4 compared to control. The Angptl4 gene encodes for a protein involved in lipid metabolism.

The Pdk4 gene encodes for a protein phosphorylating the pyruvate dehydrogenase complex, inhibiting its function to convert pyruvate to acetyl-CoA. This results in a favoring of FAs as fuel substrates in the mitochondria. Expression of Pdk4 was statistically different compared to control, but only at a relative ratio of 1.2 in mean value.

For Ucp3, the relative expression in mice fed the high-TTA diet was 1.5-fold higher and significantly different than in the controls.

FIG. 19 shows the gene expression of Angptl4, Cpt-2, Pdk4 and Ucp3 in brain after the supplementation of TTA in low (0.4%) and high (0.75%) dose in diet compared to control using the 2^{-ΔΔCt} method relative to HPRT1.

### Gene Expression Analysis—Wistar Rats

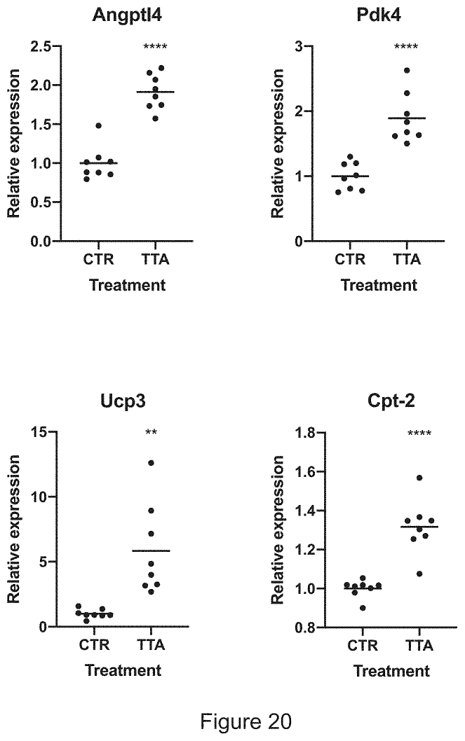

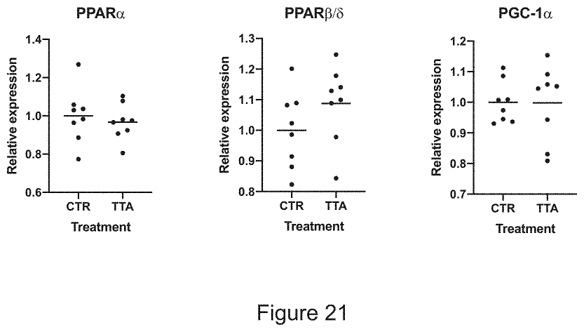

The gene expression in brains harvested from rats fed a TTA-supplemented (0.375%) diet for 50 weeks was analyzed by relative comparison to Rplp0 as a reference gene using a standard curve based on URRR. The analysis was performed by Kari Williams, and some of the results are shown in FIGS. 20 and 21.

The mean value of the relative expression of Angptl4 was approximately 2 times higher (1.9-fold) for the rats on the TTA-supplemented diet compared to the control group and significantly different. For Pdk4, the mean relative expression was also about 2 times higher (1.9-fold) and significantly different compared to the control. Ucp3 had a mean relative expression 6 times higher (5.8-fold) than controls and were also statistically different. Finally, Cpt-2 gene had a relative expression 1.3-fold higher than in the control group.

The genes for PPARα, PPARβ/δ and PGC-1α had no significant differences between the controls and treated rats (FIG. 21).

The potential long-term effects of administering TTA to rodents by analyzing the gene expression in brain. ApoE^{-/-} mice on a diet supplemented with 0.75% TTA showed an increase in the relative expression of several PPAR target genes, including Angptl4, Pdk4 and Ucp3. Similar results were obtained from the analysis of brain samples of Wistar rats fed a TTA-supplemented diet (0.375%) over 50 weeks, with an increased expression of Angptl4, Cpt-2, Pdk4 and Ucp3. However, the expression of the genes of PPARα, PPARβ/δ and the co-activator PGC-1α was not affected.

### Example 10—Effect of N-TTA and Triple-n-TTA on Body Weight and Plasma Glucose and Cholesterol Levels

Two nitrogen-supplemented fatty acids, tetradecylglycine (N-TTA or TDG)) and tetra-12-yn-1-ylglycine (triple-N-TTA) were synthetically generated as described in the examples above and their metabolic effects tested in a 4 week mouse high-fat diet study.

This animal study was conducted according to the Guidelines for the Care and Use of Experimental Animals, and the protocol was approved by the Norwegian Food Authorities. 7 to 8-weeks old male C57BL/6 mice, were obtained from Janvier Labs. After one week of acclimatization, they were divided into 3 groups of 7 animals. One group was fed a control high-fat (25 w/v) diet while the two remaining groups were fed a high-fat diet supplemented with either N-TTA (750 mg/kg body weight) or triple-N-TTA (350 mg/kg body weight). After 4 weeks of feeding, the mice were anaesthetized with Isofluorane (Forane, Abbot Laboratories Ltd, Illinois, USA) inhalation under fasting conditions, and blood and liver samples were collected.

A 250 mg liver sample was chilled on ice and homogenized in 1 mL ice-cold sucrose medium (0.25 M sucrose, 10 mM HEPES, and 1 mM Na₄EDTA, adjusted to a pH of 7.4 with KOH). The homogenates were centrifuged at 860 G for 10 min at 4° C. and the post-nuclear fraction was removed and used for further analysis. β-oxidation capacity was measured as previously described, using (1-¹⁴C) palmitoyl CoA as substrate.

Glucose and lipids in plasma was measured on the Hitachi 917 system (Roche Diagnostics, GmbH, Mannheim, Germany) using kits from Roche Diagnostics or DiaSys.

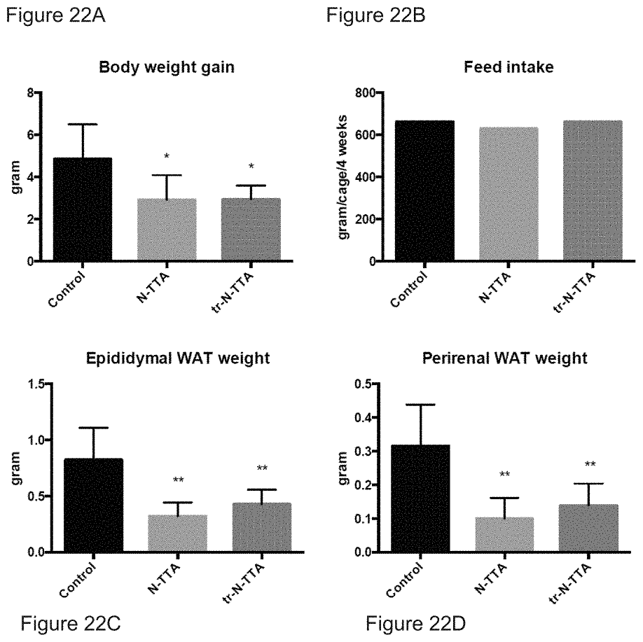

Body weight gain was significantly lower in mice supplemented with N-TTA or triple-N-TTA for 4 weeks than in mice fed a high-fat control diet (FIG. 22A). Feed intake was similar in all groups (FIG. 22B). Both epididymal and perirenal white adipose tissue depots were reduced in size by N-TTA or triple-N-TTA supplementation (FIGS. 22C and 22D).

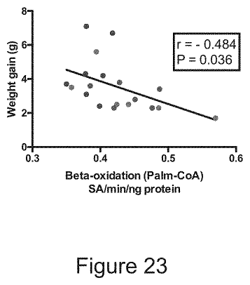

Weight gain correlated negatively to hepatic beta-oxidation as shown in FIG. 23.

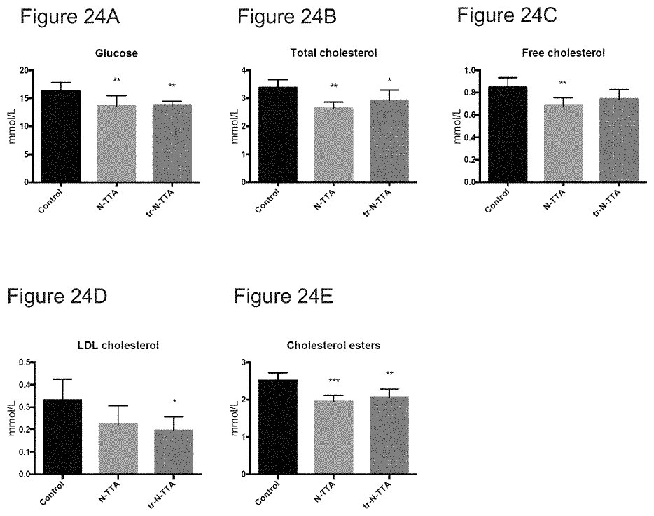

Blood glucose was significantly lower in mice supplemented with N-TTA or triple-N-TTA for 4 weeks than in mice fed a high-fat control diet (FIG. 24A). Similarly, total cholesterol, free cholesterol, LDL cholesterol and cholesterol esters were reduced by N-TTA and triple-N-TTA (FIG. 24B-24E).

Altogether, compared to control, N-TTA and triple-N-TTA reduced adipose tissue accumulation and body weight gain during 4 weeks of high-fat feeding in mice, which could be linked to increased hepatic beta-oxidation. N-TTA and triple-N-TTA also had a beneficial effect on plasma glucose and cholesterol levels.

The N-substituted fatty acids have qualitatively the same effects as the S substituted fatty acids described in the PCT applications PCT/NO99/00135, PCT/NO99/00136, PCT/NO99/00149, PCT/NO01/00082, PCT/NO01/00301, PCT/NO01/00393, PCT/NO01/00470, and PCT/GB03/02582.

1-24. (canceled)

25. A fatty acid or fatty acid containing compound for use in the prevention or treatment of a neurodegeneration disorder in an individual, wherein the fatty acid has the general formula (I):
R¹—[Xᵢ—Xᵢ]n-Y (I), wherein
R¹ is R¹ is one or more selected from a group consisting of a C₆-C₂₄ alkene with one or more double bonds or one or more triple bonds or a combination thereof, a C₆-C₂₄ alkyne, a C₆-C₂₄ alkyl substituted in one or more positions with one or more compounds selected from the group consisting of fluoride, chloride, hydroxy, C₁-C₄ alkoxy, C₁-C₄ alkylthio, C₂-C₅ acyloxy and C₁-C₄ alkyl;
n is an integer from 1 to 12;
i is an odd number and indicates the position relative to the α-carbon in Y;
at least one Xᵢ is independently selected from N, S and CH₂;
at least one Xᵢ is N or S;
Y is selected from CO—COOR₂, CH₂—COOR₂, and CH₂—R4, wherein R4 is carboxylic acid or a derivate thereof, wherein the derivate is a carboxylic ester, a glyceride or a phospholipid;
R₂, if present, is hydrogen or C1-C4 alkyl,
with the proviso that if Xᵢ is S, then one carbon-carbon triple bond or carbon-carbon double bond is positioned between the (ω-1) carbon and the (ω-2) carbon, or between the (ω-2) carbon and the (ω-3) carbon.

26. The fatty acid or fatty acid containing compound for use according to claim 25, wherein the neurodegenerative disorder is present in the individual that is afflicted with one or more of patient dementia, Alzheimer's disease, movement disorder, and Parkinson's disease.

27. The fatty acid or fatty acid containing compound for use according to claim 25, wherein said fatty acid containing compound is a glyceride derived from monoacylglycerols, diacylglycerols or triacylglycerols.

28. The fatty acid or fatty acid containing compound for use according to claim 25, wherein said fatty acid containing compound is a phospholipid derived from lysophospholipids, phosphatidylserines, phosphatidylcholines, phosphatidylethanolamines, phosphatidylinositols (P1), phosphatidic acids or phosphatidylglycerols.

29. The fatty acid or fatty acid containing compound for use according to claim 25, wherein Xᵢ is N.

30. The fatty acid or fatty acid containing compound for use according to claim 25, wherein Xᵢ is N, and R₁ is an alkyne.

31. The fatty acid or fatty acid containing compound for use according to claim 25, wherein Xᵢ is N and R₁ is an alkyne with one triple bond.

32. The fatty acid or fatty acid containing compound for use according to claim 25, wherein said compound is Tetradec-12-yn-1-ylglycine.

33. The fatty acid or fatty acid containing compound for use according to claim 25, wherein said compound is N-tetradecylglycine.

34. The fatty acid or fatty acid containing compound for use according to claim 25, wherein said compound is Tetradecylthioacetic acid.

35. The fatty acid or fatty acid containing compound for use according to claim 25, wherein the compound is 2-(tridec-12-yn-ylthio) acetic acid.

36. The fatty acid or fatty acid containing compound for use according to claim 25, wherein R¹ is a single carbon-carbon triple bond.

37. The fatty acid or fatty acid containing compound for use according to claim 25, wherein R¹ is a single carbon-carbon double bound.

38. The fatty acid or fatty acid containing compound for use according to claim 37, wherein the carbon-carbon double bond is in a cis configuration.

39. A fatty acid or fatty acid containing compound of the general formula (I):
R¹—[Xᵢ—Xᵢ]n-Y (I), wherein
R¹ is one or more selected from the group consisting of a C₆-C₂₄ alkene with one or more double bonds or with one or more triple bonds or a combination thereof; a C₆-C₂₄ alkyne; a C₆-C₂₄ alkyl substituted in one or more positions with one or more compounds selected from the group consisting of fluoride, chloride, hydroxy, C₁-C₄ alkoxy, C₁-C₄ alkylthio, C₂-C₅ acyloxy and C₁-C₄ alkyl;
n is an integer from 1 to 12;
i is an odd number and indicates the position relative to α-carbon in Y;
at least one Xᵢ is independently N or CH₂;
at least one Xᵢ is N;
Y is selected from CO—COOR₂, CH₂—COOR₂, and CH₂—R4, wherein R4 is carboxylic acid or a derivate thereof, wherein the derivate is a carboxylic ester, a glyceride or a phospholipid;
R₂, if present, is hydrogen or C1-C4 alkyl,
wherein one carbon-carbon triple bond or carbon-carbon double bond is positioned between the (ω-1) carbon and the (ω-2) carbon, or between the (ω-2) carbon and the (ω-3) carbon.

40. The fatty acid or fatty acid containing compound according to claim 39, wherein the compound is a glyceride derived from monoacylglycerols, diacylglycerols or triacylglycerols.

41. The fatty acid or fatty acid containing compound according to claim 39, wherein the compound is a phospholipid derived from lysophospholipids, phosphatidylserines, phosphatidylcholines, phosphatidylethanolamines, phosphatidylinositols (PI), phosphatidic acids or phosphatidylglycerols.

42. The fatty acid or fatty acid containing compound according to claim 39, wherein R1 is an alkyne with one triple bond.

43. The fatty acid or fatty acid containing compound according to claim 39, wherein said compound is Tetradec-12-yn-1-ylglycine, 1-N-(tridec-12-yn-yl) glycine or 2-N-(tridec-12-yn-yl) glycine.
